(19) United States
(12) Patent Application Publication
(10) Pub. No.: US 2026/0249102 A1
(43) Pub. Date: Aug. 27, 2026

(54) ACCELERATED DOSE CALCULATION USING DEEP LEARNING

(71) Applicant: Elekta (Shanghai) Technology Co., Ltd., Shanghai (CN)

(72) Inventors: Shufei Chen, Shanghai (CN); Lu Yuan, Shanghai (CN)

(21) Appl. No.: 19/165,822

(22) PCT Filed: Feb. 14, 2023
(86) PCT No.: PCT/CN2023/075823

§ 371 Date: Sep. 16, 2025

(51) Int. Cl. A61N 5/10

(52) U.S. Cl. CPC A61N 5/1031; A61N 2005/1034

(57) ABSTRACT

Systems and methods for predicting radiation dose distribution and planning a radiotherapy treatment are disclosed. An exemplary system includes a memory to store a computational model (such as a trained machine learning model), a dose prediction engine to predict a dose profile, and a treatment planning circuit. The dose prediction engine executes a dose simulation to determine a preliminary dose profile at a first statistical uncertainty level, applies the determined preliminary dose profile to the computational model to predict a refined dose profile at a second statistical uncertainty level lower than the first statistical uncertainty level. Based at least in part on refined dose profile, the treatment planning system can generate or update a radiotherapy treatment plan for use in a radiation treatment session.

### TECHNICAL FIELD

This document relates generally to dose calculation in a radiotherapy treatment system, and more particularly, to systems and methods for determining a radiation dose profile for use in radiotherapy treatment planning using artificial intelligence.

### BACKGROUND

Radiation therapy (or “radiotherapy”) may be used to treat cancers or other ailments in mammalian (e.g., human and animal) tissue. One such radiotherapy technique is provided using a linear accelerator (also referred to as “linac”), whereby a tumor is irradiated by high-energy particles (e.g., electrons, protons, ions, high-energy photons, and the like). The goal of radiation therapy is to maximize radiation dose to target tissue (e.g., tumor or other abnormal tissue) while minimizing damage to the surrounding healthy tissue, also known as organ(s) at risk (OARs). A physician prescribes a predefined amount of radiation dose to the target (tumor or other abnormal tissue) and clinical dose constraints for surrounding organs similar to a prescription for medicine. Generally, ionizing radiation in the form of a collimated beam is directed from an external radiation source toward a patient. The radiation beam can be accurately controlled to ensure the dose delivery.

A specified or selectable beam energy may be used for delivering a diagnostic energy level range or a therapeutic energy level range. Modulation of a radiation beam may be provided by one or more attenuators or collimators, such as a multi-leaf collimator (MLC) and jaws. The intensity and shape of the radiation beam can be adjusted by collimators to avoid damaging healthy tissue adjacent to the targeted tissue, such as by conforming the projected beam to a profile of the targeted tissue.

Treatment planning generally involves determining radiotherapy parameters for implementing a treatment goal under the constraints. Examples of the radiotherapy parameters include radiation beam angles, radiotherapy dose intensity, dose distribution or profile, etc. The dose distribution or profile can be calculated using a dose calculation algorithm. The outcome of the treatment planning process is a radiotherapy treatment plan, hereinafter also referred to as a “treatment plan”. The treatment plan can be developed using a software noted as treatment planning system (TPS) before radiotherapy delivery using one or more medical imaging techniques, such as images from X-rays, computed tomography (CT), nuclear magnetic resonance (MR), positron emission tomography (PET), single-photon emission computed tomography (SPECT), or ultrasound, among others. A clinician may use images of patient anatomy to identify target tumors and surrounding organs near the tumor, delineate the target that is to receive prescribed radiation dose, and similarly delineate nearby tissue such as organs at risk (OARs) of damage from the radiation treatment. The delineation can be done manually, or by using an automated tool that assists in identifying or delineating the target tumor and OARs. A radiotherapy treatment plan can then be generated using an optimization technique based on clinical and dosimetric objectives and constraints (e.g., the maximum, minimum, mean, and a fraction of dose to a fraction or the whole tumor volume, and similar measures for the critical organs).

Accurate dose calculation is important for the modern radiotherapy techniques, such as intensity-modulated radiation therapy (IMRT) and volumetric modulated arc therapy (VMAT). Monte Carlo (MC) simulation is an algorithm widely used in medical physics, and has been used to calculate dose distribution or profile. MC simulation is based on a statistical model that calculates the dose distribution given a limited set of particle interaction types and their probabilities. The probability for a certain interaction is given by the linear attenuation coefficient. Accuracy of the MC simulation is largely dependent on the number of particles in the simulation. Statistical uncertainty of MC-based dose calculation decreases as the number of simulated particles per voxel is increased.

### OVERVIEW

MR-linac is a radiation treatment system that combines linac radiotherapy with diagnostic-level magnetic resonance imaging (MRI). The MR-linac can enable in-room MRI for anatomic and physiological treatment adaptation and response monitoring, and has a potential to reduce treatment margins with real-time visualization and target tracking. Tumors and surrounding tissue can be precisely located, their movement tracked, and treatment adapted in real time in response to changes in tumor position, shape, biology and spatial relationship to critical organs at the time of treatment.

The treatment planning process may include using a three-dimensional (3D) image of the patient to identify a target region (e.g., the tumor) and to identify critical organs near the tumor. Creation of a treatment plan can be a time-consuming process where a planner tries to comply with various treatment objectives or constraints (e.g., dose-volume histogram (DVH), overlap volume histogram (OVH)), taking into account their individual importance (e.g., weighting) in order to produce a treatment plan that is clinically acceptable. The treatment plan can be comprised of numerical parameters that specify the direction, cross-sectional shape, and intensity of each radiation beam. Once generated, the treatment plan can be executed by positioning the patient in the treatment machine and delivering the prescribed radiation therapy directed by the optimized plan parameters. In some examples, the radiotherapy treatment plan may include dose “fractioning,” whereby a sequence of radiation treatments may be provided over a predetermined period of time (e.g., 30-45 daily fractions), with each treatment including a specified fraction of a total prescribed dose. During treatment, the position of the patient and the position of the target tumor in relation to the treatment machine (e.g., linac) is very important in order to ensure the target tumor and not healthy tissue is irradiated.

Monte Carlo (MC) algorithm has been regarded as a standard method of calculating radiation dose distribution. The accuracy of dose distribution calculated using MC-based dosimetry algorithm, however, relies on sufficient repeated random sampling of particles (e.g., photons or electrons) in the simulation. In a treatment planning system (TPS), the number of particles (also referred to as “histories”) are related to the statistical uncertainty (also known as statistical fluctuations) of final dose distribution. The statistical uncertainty of dose calculation, generally represented by a percentage, reflects the accuracy and robustness of dose calculation. As the number of histories (i.e., simulated particles per voxel) is increased, the statistical uncertainty of dose distribution decreases. Accordingly, to achieve a highly accurate and robust dose calculation at a clinically acceptable low-uncertainty level (e.g., less than 5%, or no more than 1%) using the conventional MC-based dosimetry algorithm, it generally requires intensive computation, which may take significant amount of computation time. For example, in a prostate VMAT plan, the per segment dose computation time (T_{seg}) is <0.1 second at an uncertainty level of 30% per control point. The computation time T_{seg} increases dramatically as the accuracy requirement increases (i.e., lower statistical uncertainty levels). For example, T_{seg} reaches approximately 0.37 second at a 10% uncertainty level, approximately 3.7 seconds at a 3% uncertainty level, or approximately 33.1 seconds at a 1% uncertainty level. For a treatment plan of 90 segments of the prostate, the total dose calculation time can be up to 336 seconds at a 3% uncertainty level, and 2975 seconds at a 1% uncertainty level. Furthermore, in certain implementations where the MC dose calculation is integrated into a treatment planning optimization process, repeated dose calculation may be required. This may further increase the computation time and reduce the efficiency of the optimization process. For at least the above reasons, the present inventors have recognized an unmet need for more efficient techniques that can determine a dose distributions with high accuracy at sufficiently low and clinically acceptable statistical uncertainties, but take much less computation time than the conventional MC-based dosimetry methods.

The present document discusses systems and methods for determining a radiation dose profile and planning a radiotherapy treatment using the determined dose profile. An exemplary system includes a memory to store a computational model (such as a trained machine learning model), a dose prediction engine to predict a dose profile, and a treatment planning circuit. The dose prediction engine can execute a dose simulation to determine a preliminary dose profile at a first statistical uncertainty level, applies the determined preliminary dose profile to the computational model to predict a refined dose profile at a second statistical uncertainty level lower than the first statistical uncertainty level. Based at least in part on refined dose profile, the treatment planning system can generate a radiotherapy treatment plan for use in a radiation treatment session of the patient. Optionally, the radiation dose profile is determined using artificial intelligence (AI) based techniques.

Example 1 is a system for providing radiotherapy to a patient according to a treatment plan, the system comprising: a memory configured to store a computational model; a dose prediction engine configured to: execute a dose simulation to calculate a preliminary dose profile at a first statistical uncertainty level of dose calculation; and apply the calculated preliminary dose profile to the computational model to predict a refined dose profile at a second statistical uncertainty level, the second statistical uncertainty level being lower than the first statistical uncertainty level; and a treatment planning system configured to generate or update a radiotherapy treatment plan based at least in part on the predicted refined dose profile. Optionally, the treatment plan is an artificial intelligence (AI)-based treatment plan.

In Example 2, the subject matter of Example 1 optionally includes the preliminary dose profile and the predicted refined dose profile each including respective dose images representing spatial dose distributions at a radiotherapy treatment area and a treatment exclusion area.

In Example 3, the subject matter of any one or more of Examples 1-2 optionally includes the preliminary dose profile and the predicted refined dose profile each represented by at least one of: a percentage depth dose (PDD) curve; a radial dose curve; a dose-volume histogram; an overlap volume histogram; or a three-dimensional dose distribution.

In Example 4, the subject matter of any one or more of Examples 1-3 optionally includes the dose prediction engine that can be configured to predict the refined dose profile further using patient information including at least one of a particle density or a planning target volume (PTV) structure.

In Example 5, the subject matter of any one or more of Examples 1-4 optionally includes the dose prediction engine that can be configured to calculate the preliminary dose profile using (i) patient anatomical data corresponding to a mapping of at least a radiotherapy treatment area and (ii) a dose calculation algorithm.

In Example 6, the subject matter of Example 5 optionally includes the dose calculation algorithm that can include a Monte Carlo (MC) algorithm or a collapsed cone convolution (CCC) algorithm.

In Example 7, the subject matter of any one or more of Examples 5-6 optionally includes, wherein: the dose prediction engine is configured to calculate the preliminary dose profile using an initial dose radiotherapy treatment plan; and the treatment planning system is configured to update the initial radiotherapy treatment plan when the predicted refined dose profile satisfies a dose criterion.

In Example 8, the subject matter of any one or more of Examples 1-7 optionally includes the computational model that can be include a trained deep-learning (DL) model, the system further comprising a training module configured to: construct training data obtained from prior dose simulations on a radiotherapy treatment area, the training data comprising (i) a first set of dose profiles at the first statistical uncertainty level, and (ii) a second set of dose profiles at the second statistical uncertainty level lower than the first statistical uncertainty level; and generate the trained DL model using the constructed training data, the trained DL model representing an established mapping from the first set of dose profiles to the second set of dose profiles for the same radiotherapy treatment area, wherein the dose prediction engine is configured to apply the preliminary dose profile to the trained DL model to predict the refined dose profile.

In Example 9, the subject matter of Example 8 optionally includes the trained DL model that can include a convolutional neural network (CNN) with a U-Net architecture, the U-Net architecture including at least one of: a standard U-Net; a Hierarchy Dense (HD) U-Net; an Attention U-Net; a Group Normalization U-Net; or a Recurrent Residual U-Net.

In Example 10, the subject matter of any one or more of Examples 8-9 optionally includes the training module that can be configured to evaluate a model performance during model training, and to generate the trained DL model in response to the model performance satisfying a specific criterion, the model performance including at least one of: a dose difference metric; a radiotherapy planning target volume (PTV) dose coverage metric; a gamma passing ratio; or a structure similarity.

In Example 11, the subject matter of any one or more of Examples 8-10 optionally includes the training data that further comprises a third set of dose profiles obtained from the prior dose simulations on the radiotherapy treatment area, the a third set of dose profiles at a third statistical uncertainty level different from the first statistical uncertainty level and higher than the second statistical uncertainty level, wherein the training module is configured to aggregate the first set of dose profiles with the third set of dose profiles, and to generate the trained DL model using the constructed training data including the aggregated first and third sets of dose profiles, the trained DL model representing an established mapping from the aggregated first and third sets of dose profiles to the second set of dose profiles.

In Example 12, the subject matter of Example 11 optionally includes, wherein to predict the refined dose profile, the dose prediction engine is configured to: execute a dose stimulation to determine a first preliminary dose profile at the first statistical uncertainty level and a second preliminary dose profile at the third statistical uncertainty level; aggregate the first preliminary dose profile with the second preliminary dose profile; and apply the aggregated first and second preliminary dose profiles to the trained DL model to predict the refined dose profile at the second statistical uncertainty level.

In Example 13, the subject matter of any one or more of Examples 8-12 optionally includes the training data that further comprises an intermediate set of dose profiles obtained from the prior dose simulations on the radiotherapy treatment area at an intermediate statistical uncertainty level lower than the first statistical uncertainty level and higher than the second statistical uncertainty level, wherein the training module is configured to generate the trained DL model using the constructed training data including the intermediate set of dose profiles, the trained DL model including (i) a first trained DL model being trained to establish a mapping from the first set of dose profiles to the intermediate set of dose profiles and (ii) a second trained DL model being trained to establish a mapping from the intermediate set of dose profiles to the second set of dose profiles.

In Example 14, the subject matter of Example 13 optionally includes, wherein to predict the refined dose profile, the dose prediction engine is configured to: apply the preliminary dose profile to the first trained DL model to predict an intermediate dose profile at the intermediate statistical uncertainty level; and apply the predicted intermediate dose profile to the second trained DL model to predict the refined dose profile at the second statistical uncertainty level.

In Example 15, the subject matter of any one or more of Examples 1-14 optionally includes the dose prediction engine that can be configured to: pre-process the preliminary dose profile including to down-sample, or to truncate at least a portion of, the preliminary dose profile; and apply the pre-processed the preliminary dose profile to the computational model to predict the refined dose profile.

In Example 16, the subject matter of any one or more of Examples 1-15 optionally includes the dose prediction engine that can be configured to post-process the predicted refined dose profile including to up-sample, or to interpolate or extrapolate at least a portion of, the predicted refined dose profile.

In Example 17, the subject matter of any one or more of Examples 1-16 optionally includes a user interface configured to present the predicted refined dose profile or the generated or updated radiotherapy treatment plan to the user.

In Example 18, the subject matter of any one or more of Examples 1-17 optionally includes a radiotherapy device configured to deliver a radiotherapy to the patient in accordance with the generated or updated radiotherapy treatment plan.

Example 19 is a method of providing radiotherapy according to a treatment plan, the method comprising: calculating a preliminary dose profile at a first statistical uncertainty level of dose calculation in a dose simulation; predicting a refined dose profile at a second statistical uncertainty level by applying the calculated preliminary dose profile to a computational model, the second statistical uncertainty level being lower than the first statistical uncertainty level; and generating or updating a radiotherapy treatment plan based at least in part on the predicted refined dose profile. Optionally, the treatment plan is an artificial intelligence (AI)-based treatment plan.

In Example 20, the subject matter of Example 19 optionally includes the preliminary dose profile and the predicted refined dose profile each including respective dose images representing spatial dose distributions at a radiotherapy treatment area and a treatment exclusion.

In Example 21, the subject matter of any one or more of Examples 19-20 optionally includes determining the preliminary dose profile includes using (i) patient anatomical data corresponding to a mapping of at least a radiotherapy treatment area and (ii) a dose calculation algorithm.

In Example 22, the subject matter of Example 21 optionally includes determining the preliminary dose profile that can further include using an initial dose radiotherapy treatment plan, wherein generating or updating the radiotherapy treatment plan includes updating the initial radiotherapy treatment plan when the predicted refined dose profile satisfies a dose criterion.

In Example 23, the subject matter of any one or more of Examples 19-22 optionally includes the computational model that can include a trained deep-learning (DL) model, the method further comprising: constructing training data obtained from prior dose simulations on a radiotherapy treatment area, the training data comprising (i) a first set of dose profiles at the first statistical uncertainty level, and (ii) a second set of dose profiles at the second statistical uncertainty level lower than the first statistical uncertainty level; and training a DL model using the constructed training data, the trained DL model representing an established mapping from the first set of dose profiles to the second set of dose profiles for the same radiotherapy treatment area, wherein predicting the refined dose profile includes applying the calculated preliminary dose profile to the trained DL model.

In Example 24, the subject matter of Example 23 optionally includes evaluating a model performance, and generating the trained DL model in response to the model performance satisfying a specific criterion, the model performance including at least one of: a dose difference metric; a radiotherapy planning target volume (PTV) dose coverage metric; a gamma passing ratio; or a structure similarity.

In Example 25, the subject matter of any one or more of Examples 23-24 optionally includes trained DL model that can include a convolutional neural network (CNN) with a U-Net architecture including at least one of a standard U-Net, a Hierarchy Dense (HD) U-Net, an Attention U-Net, a Group Normalization U-Net, or a Recurrent Residual U-Net.

In Example 26, the subject matter of any one or more of Examples 23-25 optionally includes the training data that further comprises a third set of dose profiles obtained from the prior dose simulations on the radiotherapy treatment area, the third set of dose profiles at a third statistical uncertainty level different from the first statistical uncertainty level and higher than the second statistical uncertainty level, the method further comprising: aggregating the first set of dose profiles with the third set of dose profiles; and training the DL model using the constructed training data including the aggregated first and third sets of dose profiles, the trained DL model representing an established mapping from the aggregated first and third sets of dose profiles to the second set of dose profiles.

In Example 27, the subject matter of Example 26 optionally includes predicting the refined dose profile at the second statistical uncertainty level that can include: executing a dose stimulation to determine a first preliminary dose profile at the first statistical uncertainty level and a second preliminary dose profile at the third statistical uncertainty level; aggregating the first preliminary dose profile with the second preliminary dose profile; and applying the aggregated first and second preliminary dose profiles to the trained DL model to predict the refined dose profile.

In Example 28, the subject matter of any one or more of Examples 23-27 optionally includes the training data that further comprises an intermediate set of dose profiles obtained from the prior dose simulations on the radiotherapy treatment area at an intermediate statistical uncertainty level lower than the first statistical uncertainty level and higher than the second statistical uncertainty level, wherein training the DL model includes training a first DL model to establish a mapping from the first set of dose profiles to the intermediate set of dose profiles, and training a second DL model to establish a mapping from the intermediate set of dose profiles to the second set of dose profiles.

In Example 29, the subject matter of Example 28 optionally includes predicting the refined dose profile at the second statistical uncertainty level that can include: applying the preliminary dose profile to the first trained DL model to predict an intermediate dose profile at the intermediate statistical uncertainty level; and applying the predicted intermediate dose profile to the second trained DL model to predict the refined dose profile at the second statistical uncertainty level.

In Example 30, the subject matter of any one or more of Examples 19-29 optionally includes delivering a radiotherapy in accordance with the generated or updated radiotherapy treatment plan using a radiotherapy device.

The AI-based dose prediction in accordance with various examples discussed in this document may improve the efficiency and accuracy of dose calculation at a clinically acceptable low-uncertainty level and the quality of a radiation treatment plan. The AI-based dose prediction predicts a refined dose distribution with a much lower statistical uncertainty (and a higher accuracy) using a pre-calculated preliminary dose distribution with a higher statistical uncertainty (or less accuracy). The preliminary dose distribution, when calculated using conventional MC algorithm or other dose calculation algorithms, requires much less computational time. The prediction process, which can be based on a pre-trained model (e.g., a deep learning model), add little overhead of time and system complexity. As a result, the AI-based dose prediction discussed herein can provide highly accurate dose distributions with sufficiently low and clinically acceptable statistical uncertainties but takes substantially less computational time and cost. Consequently, the overall cost savings for treatment planning and improved radiotherapy can be achieved.

The above is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an exemplary radiotherapy system.

FIG. 2A illustrates an exemplary radiotherapy system that can provide a therapy beam.

FIG. 2B illustrates an exemplary combined system including a computed tomography (CT) imaging system and a radiation therapy system.

FIG. 3 illustrates a partially cut-away view of an exemplary combined system including a nuclear magnetic resonance (MR) imaging system and a radiation therapy system.

FIGS. 4A-4C illustrate examples of AI-based dose prediction such as using the dose engine of the radiotherapy system as shown in FIG. 1.

FIG. 5 illustrates an exemplary process for training a deep learning (DL) network model to predict a refined low-uncertainty dose profile using a pre-calculated preliminary, high-uncertainty dose profile.

FIGS. 6A-6E illustrate examples of CNN models with a U-Net architecture or a variant of U-Net architecture that can be trained to predict a refined low-uncertainty dose profile using a preliminary high-uncertainty dose profile.

FIGS. 7-8 illustrate exemplary methods of predicting a radiation dose profile using AI-based techniques, and using the predicted dose profile in a radiotherapy treatment planning process or a secondary dose check process.

FIG. 9 illustrates generally a block diagram of an example machine upon which any one or more of the techniques (e.g., methodologies) discussed herein may perform.

### DETAILED DESCRIPTION

This disclosure describes systems and methods for predicting a radiation dose profile and planning a radiotherapy treatment based at least one the dose profile prediction. An exemplary system includes a memory to store a computational model (such as a trained machine learning model), a dose prediction engine to predict a dose profile, and a treatment planning circuit. The dose prediction engine can execute a dose simulation to determine a preliminary dose profile at a first statistical uncertainty level, and apply the preliminary dose profile to the computational model to determine a refined dose profile at a second statistical uncertainty level lower than the first statistical uncertainty level. Based at least in part on refined dose profile, the treatment planning system can generate or update a radiotherapy treatment plan for use in a radiation treatment session.

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized and that structural, logical and electrical changes may be made without departing from the scope of the present disclosure. The following detailed description is, therefore, not be taken in a limiting sense, and the scope of the present disclosure is defined by the appended aspects and their equivalents.

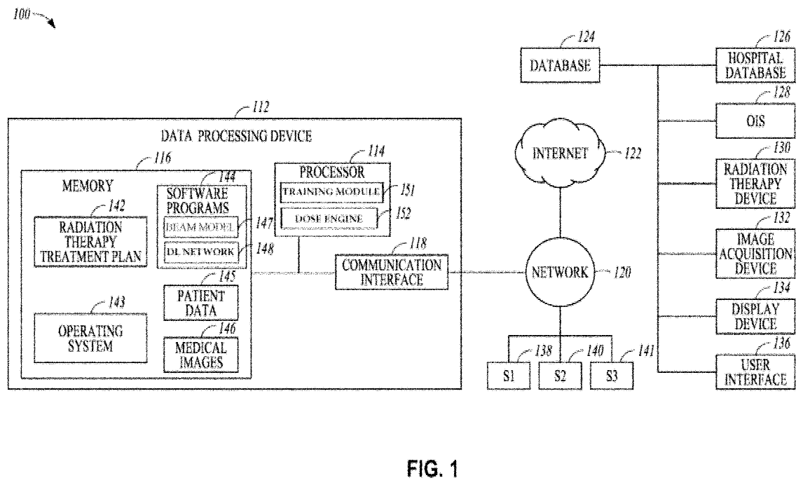

FIG. 1 illustrates an exemplary radiotherapy system 100 for providing radiation therapy to a patient. The radiotherapy system 100 includes, among other components, a data processing device 112. The data processing device 112 may be connected to a network 120. The network 120 may be connected to the Internet 122. The network 120 can connect the data processing device 112 with one or more of a database 124, a hospital database 126, an oncology information system (OIS) 128, a radiation therapy device 130, an image acquisition device 132, a display device 134, and a user interface 136. The data processing device 112 can be configured to generate or update radiotherapy treatment plans 142 to be used by the radiation therapy device 130.

The data processing device 112 may include a memory 116, a processor 114, and a communication interface 118. The memory 116 may store computer-executable instructions, such as a radiotherapy treatment plan 142 (e.g., original treatment plans, adapted treatment plans and the like), an operating system 143, software programs 144, and any other computer-executable instructions to be executed by the processor 114. The memory 116 may additionally store data, such as medical images 146, patient data 145, and other data required to implement a radiotherapy treatment plan 142.

The software programs 144 may include one or more software packages that, when executed by a machine such as the processor 114, can perform specific image processing and generating a radiation treatment plan 142 or updating an existing treatment plan. In an example, the software programs 144 can convert medical images of one format (e.g., MRI) to another format (e.g., CT) by producing synthetic images, such as pseudo-CT images. For instance, the software programs 144 may include image processing programs to train a predictive model for converting a medical image from the medical images 146 in one modality (e.g., an MR image) into a synthetic image of a different modality (e.g., a pseudo CT image); alternatively, the trained predictive model may convert a CT image into an MR image. In another example, the software programs 144 may register the patient image (e.g., a CT image or an MR image) with that patient's dose distribution (which can also be represented as an image) so that corresponding image voxels and dose voxels are associated appropriately by the network. In yet another example, the software programs 144 may substitute functions of the patient images such as signed distance functions or processed versions of the images that emphasize some aspect of the image information. Such functions might emphasize edges or differences in voxel textures, or any other structural aspect useful to neural network learning. The software programs 144 may substitute functions of the dose distribution that emphasize some aspect of the dose information. Such functions might emphasize steep gradients around the target or any other structural aspect useful to neural network learning.

In an example, the software programs 144 may generate projection images for a set of two-dimensional (2D) and/or 3D CT or MR images depicting an anatomy (e.g., one or more targets and one or more OARs) representing different views of the anatomy from the treatment gantry angles of the radiotherapy equipment. For example, the software programs 144 may process the set of CT or MR images and create a stack of projection images depicting different views of the anatomy depicted in the CT or MR images from various perspectives of the gantry of the radiotherapy equipment. In particular, one projection image may represent a view of the anatomy from 0 degrees of the gantry, a second projection image may represent a view of the anatomy from 45 degrees of the gantry, and a third projection image may represent a view of the anatomy from 90 degrees of the gantry. The degrees may be directions of the beams relative to a particular axis of the anatomy depicted in the CT or MR images. The axis may remain the same for each beam of the different degrees.

In an example, the software programs 144 may generate graphical aperture image representations of MLC leaf positions at various gantry angles. These graphical aperture images are also referred to as aperture images. In particular, the software programs 144 may receive a set of control points that are used to control a radiotherapy device to produce a shaped radiotherapy beam. The control points may represent the beam intensity, gantry angle relative to the patient position, and the leaf positions of the MLC, among other machine parameters. Based on these control points, a graphical image may be generated to graphically represent the beam shape and intensity that is output by the MLC and jaws at each particular gantry angle. The software programs 144 may align each graphical image of the aperture at a particular gantry angle with the corresponding projection image at that angle that was generated. The images are aligned and scaled with the projections such that each projection image pixel is aligned with the corresponding aperture image pixel.

The software programs 144 may include a treatment planning software. The treatment planning software, when executed such as by a treatment planning system (TPS), can generate the radiotherapy treatment plan 142 using one or more of the medical images 146. In an example, execution of the treatment planning software can produce a graphical aperture image representation of MLC leaf positions at a given gantry angle for a projection image of the anatomy representing the view of the anatomy from the given gantry angle.

As depicted, the software programs 144 may include a beam model 147. The beam model is represented by various characteristics of radiation beams with all the imports of a broad radiation field specific to a treatment machine and exiting the radiation machine and impinging upon the patient. Using an appropriately determined beam model, machine parameters or control points for a given type of machine can be calculated, and the radiation machine can output a beam from the MLC that achieves the same or similar estimated graphical aperture image representation of the MLC leaf positions and intensity. The treatment planning software, when executed, may output an image representing an estimated image of the beam shape and the intensity for a given gantry angle and for a given projection image of the gantry at that angle, and the function may compute the control points for a given radiotherapy device to achieve that beam shape and intensity.

The beam model 147 can be represented by a function of one or more beam model types that characterize various properties of one or more radiation modality, such as a photon or an electron. Different beam models may differ in the number and/or configuration of the radiation sources. As such, beam model parameters (e.g., size, position, energy spectrum, or fluence distribution of a radiation source) may vary from one beam model type to another. By way of example and not limitation, the beam model parameters may include size and position of one or more photon sources within the radiation machine, maximum or average energy of a photon spectrum for photons emitted from the radiation machine, factors describing the shape of a photon spectrum emitted from the radiation machine, size and position of one or more electron sources within the radiation machine, maximum or average energy of an electron spectrum emitted from the radiation machine, factors describing the shape of an electron spectrum, or one or more numbers describing how radiation (e.g., electrons or photons) emitted by the radiation machine can vary off-axis, among others.

In an example, the beam model 147 is a full Monte Carlo (MC) model. The full MC model can generally produce accurate dose calculation by simulating primary and scattered photons and contamination electrons from the linac head. The full MC model thus can handle complex beam arrangements associated with modern radiotherapy techniques, such as stereotactic body radiotherapy (SBRT), intensity modulated radiotherapy (IMRT), or volumetric modulated arc therapy (VMAT). However, the full MC model can involve a large number of machine components. Detailed modeling of each of the components may require large phase space files (PSFs) to contain vast amount of particle information such as energy, position, direction, charge, regions of creation, or interaction of particles at different scoring planes. Another example of the beam model 147 is a virtual source model (VSM), which can approximate a full MC model. An MC-based dosimetry algorithm provides the ability to accurately simulate dose distributions within heterogeneous media and thus for clinical situations in radiation therapy. A VSM may include two virtual photon sources (representing the contribution from target and flattening filter) and one virtual electron source. Each virtual source of the VSM can generate particle distributions comparable to (with the accuracy required) the dose distribution (e.g., energy and angular distributions) from the original PSFs of the MC model. Radiation beams emitted from a radiation source and transport through a treatment head (e.g., a linac head) can be modelled by a beam model, such as a VSM. Radiation transport through the MLCs and jaws can be modelled by transmission filters. Medical images 146 (e.g., a CT image) can be produced by the radiation out of the MLC and jaws. From the medical image 146, dose profiles (e.g., dose metrics, characteristics, distributions, or dose images, in various data formats) can be calculated based on electron density of the tissue, such as using a dose engine 152 in the processor 114. Various dose calculation algorithms, based on Monte Carlo techniques, may be used to calculate the dose profiles, including, for example, voxel Monte Carlo (VMC), or X-ray voxel Monte Carlo (XVMC).

The accuracy of dose calculation can be dependent upon sufficient repeated random sampling of particles in a MC simulation. The number of particles involved in the simulation determines the statistical uncertainty of final dose distribution. As the number of histories (i.e., simulated particles per voxel) is increased, the statistical uncertainty of the dose distribution decreases. As such, intensive computation and long computation time are generally required in MC-based dose calculations in order to achieve a clinically acceptable low statistical uncertainty (e.g., 1%) level. An AI-based dosimetry algorithm can be implemented in the beam model 147 to predict a refined dose profile at a low-uncertainty level (e.g., 1%) using a pre-calculated preliminary dose profile at a much higher statistical uncertainty (e.g., 30%). Because the preliminary high-uncertainty dose profile can be computed using significantly less time, the time taken for predicting the refined dose profile can be reduced, and the overall dose calculation efficiency can be improved.

The preliminary dose profile and the refined dose profile may each be represented by respective radiotherapy dose distribution images (or simply “dose images”). A dose image can take the form of a two-dimensional (2D) or three-dimensional (3D) dose matrix representing a spatial distribution of dose data on at least one treatment area and optionally further at least one treatment exclusion area. The dose image may comprise dose data of a color image. Alternatively, the dose image may comprise dose data of a grayscale image. In some examples, a dose image can be produced from linear interpolation or nearest neighbor interpolation of archived dose data at a lower resolution; or the radiotherapy dose data comprises an indication of an amount of radiotherapy treatment at the coordinates within the coordinate space of the anatomical area. In some examples, the preliminary dose profile and the refined dose profile each may include a dose metric representing a statistical distribution of dose data. Examples of the dose metric may include a percentage depth dose (PDD) curve that characterizes relative dose quantity determined as the ratio between the axis dose at a specific depth and the axis dose at a reference dose depth, a dose profile that characterizes off-axis dose distribution (e.g., doses at diagonals), a percentage radial dose (PRD) curve representing changes of relative dose with a radial distance; a dose-volume histogram, an overlap volume histogram, or a three-dimensional dose distribution. In this document, the various dose metrics, characteristics, distributions, or dose images, in various data formats, are collectively referred to as “dose profiles.”

The software programs 144 may use a computational model to predict values for one or more model parameters of the beam model 147. In an example, the computational model can include a machine learning model, such as a trained deep learning (DL) model 148. The trained DL model 148 can be such trained that it can predict a refined dose profile at a specific low statistical uncertainty (e.g., 1%) using a pre-calculated preliminary dose profile at a higher statistical uncertainty (e.g., 30%, 20%, or 10%). In addition to the preliminary dose profile, other data can be provided to the trained DL model 148 to predict the refined dose profile, including the patient data 145 or data of other medical images 146. The dose engine 152 can calculate the preliminary dose profile in dose simulations, such as using the medical images 146 and/or the patient data 145. In an example, patient anatomical data corresponding to a mapping of at least a radiotherapy treatment area, optionally further of a treatment exclusion area, can be used to calculate the preliminary dose profile. The preliminary dose profile may be calculated using a MC algorithm, a Collapsed Cone Convolution (CCC) algorithm, or other dose calculation algorithms. Such algorithms can simulate particle interactions and probabilities of such interactions among particles (e.g., photons or electrons).

The trained DL model 148 may have a multi-layered network architecture comprising an input layer, an output layer, and a large number (e.g., dozens or hundreds) of layers, of hidden layers between the input and output layers that are arranged or interconnected in complex ways. The trained DL model 148, when properly trained, can produce human level performance on, for example, image recognition tasks. In addition to weighted sums of inputs, some layers compute other operations on the prior layer outputs such as convolution. Convolutions and the filters derived from them can locate edges in images, or temporal/pitch features in sound streams, and succeeding layers find larger structures composed of these primitives. Such trained DL network model which involve the use of convolutional layers are referred to as convolutional neural networks (CNNs). A CNN network can automatically learn the characteristics of data from samples, eliminating the complex feature extraction in conventional machine learning models which generally requires substantial expert knowledge. Additional advantage of the CNN is that by means of weight sharing, the scale of CNN parameters can be greatly reduced. As a result, the complexity of the training process can be reduced, the converging speed can be increased, and the model generalization ability can be enhanced. Other examples of the trained DL model 148 may include a recurrent neural network (RNN), a deep belief network (DBN), or a hybrid neural network comprising two or more neural network models of different types or different model configurations. An RNN include connections between nodes to form a directed graph along a temporal sequence. It can use internal state (memory) to store past information, and the network decisions are influenced by what it has learnt from the past. In an example, a long short-term memory (LSTM) network may be used. The LSTM is a type of RNN architecture, characterized by feedback connections. A common LSTM unit can be composed of a cell, an input gate to decide how much new information is to be added to the cell, a forget gate to decide what information is to be discarded or preserved in the cell, and an output gate to decide the values to output.

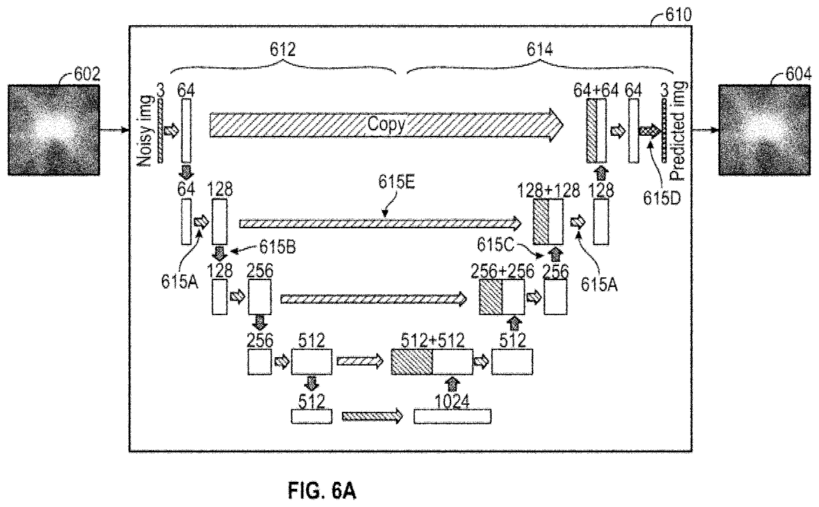

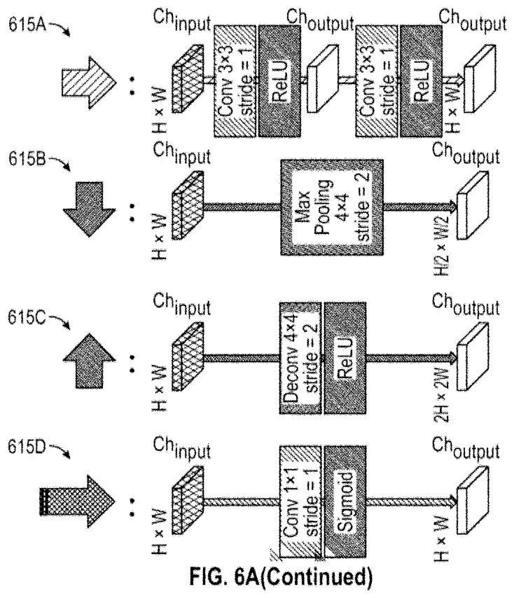

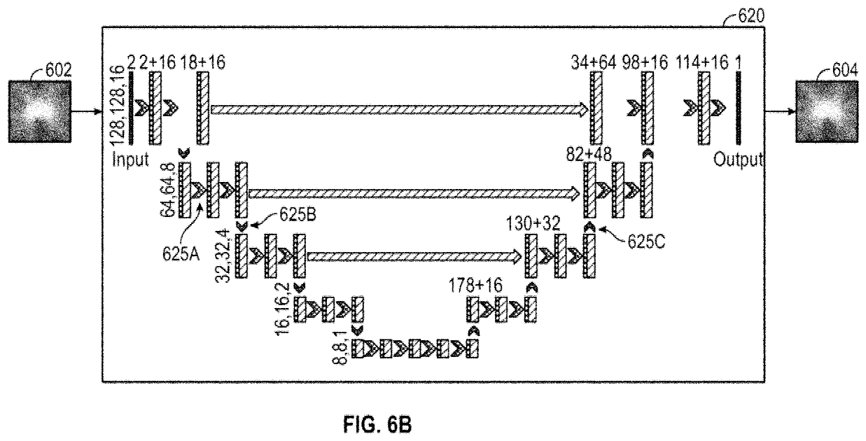

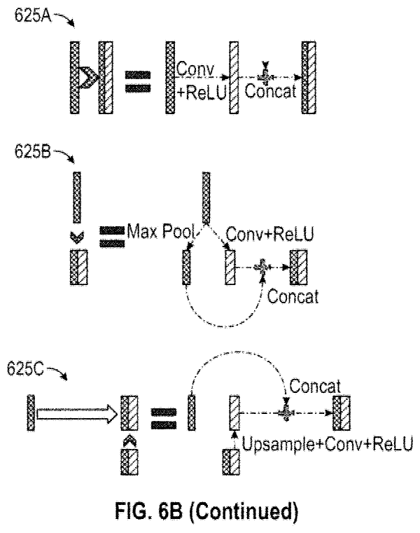

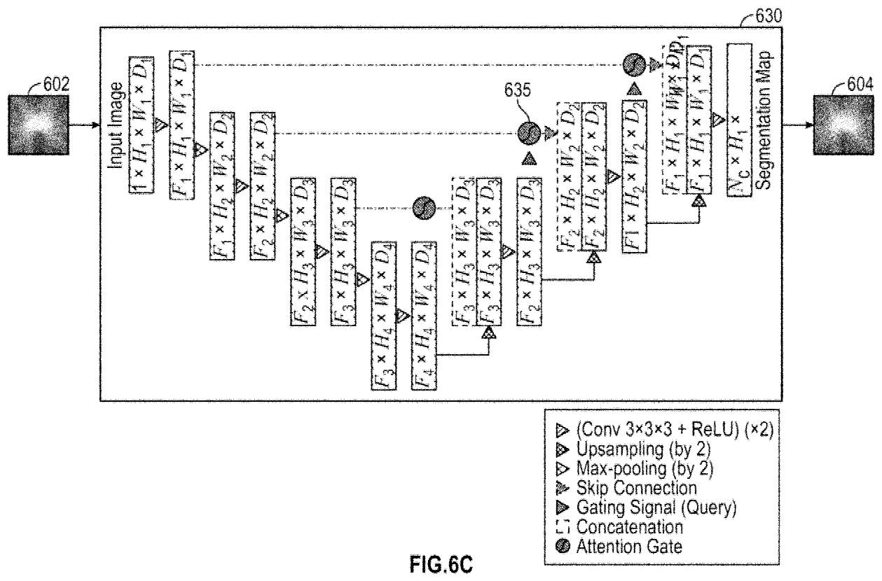

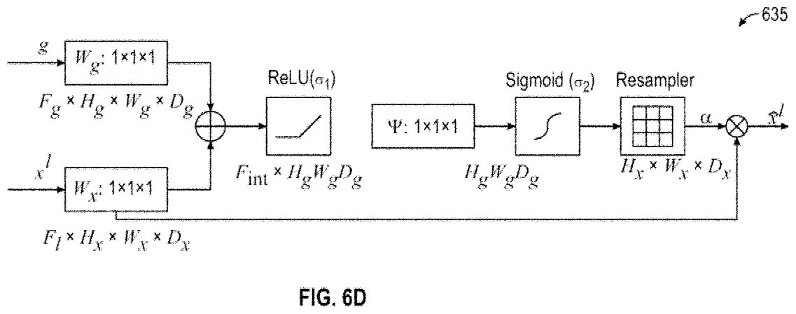

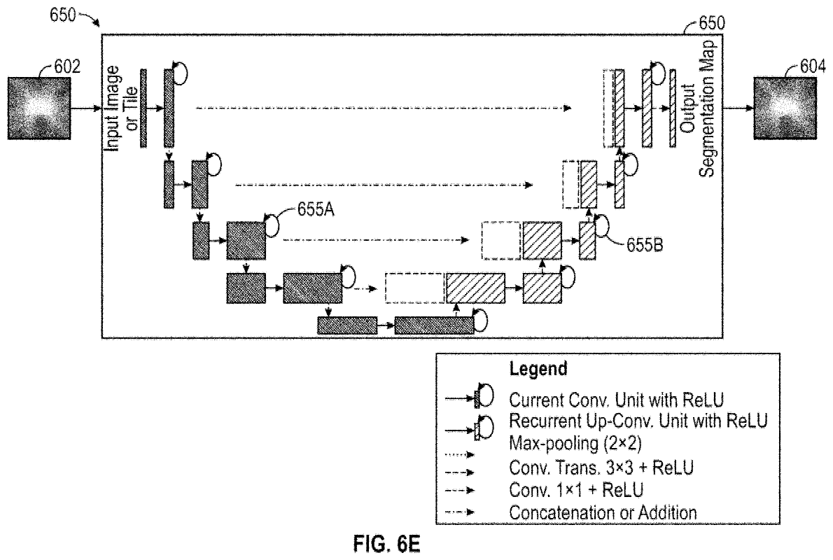

Some CNNs include skip connections that can splice nodal data at one level of a network with that of nodes at another level. The inclusion of the skip connections can improve accuracy and shorten model training. An important example is the U-Net architecture. The U-Net is a deep encoder-decoder model that has been used for digital image denoising and image segmentation. The U-Net generally consists of an encoder with convolution layers, and a decoder with up-convolution layers. The outputs of the encoder can be concatenated to the inputs of the decoder in each depth. The combination of encoded and decoded features across the same network hierarchy levels can lead to more accurate image segmentation or classification. Variants of the U-Net use different structures other than down-sampling and up-sampling layer to get potential information besides standard U-Net architecture. Examples of the U-Net variants include a Hierarchy Dense U-Net (a U-Net with densely connected convolutional layers and densely connected down-sampling layers), an Attention U-Net (a U-Net with additive attention gates to selectively enhance certain portions of the image), a Group Normalization U-Net (a U-Net with normalization layers that divide the features into certain groups and normalizes the features within each group separately), or a Residual U-Net (a U-Net with recurrent convolutional layers), among others. Examples of the U-Net architecture and its variants are described further below with reference to FIGS. 6A-6E.

In addition to the memory 116 storing the software programs 144, the software programs 144 may additionally or alternatively be stored on a removable computer medium, such as a hard drive, a computer disk, a CD-ROM, a DVD, a HD, a Blu-Ray DVD, USB flash drive, a SD card, a memory stick, or any other suitable medium; and the software programs 144 when downloaded to data processing device 112 may be executed by processor 114.

The processor 114 may be communicatively coupled to the memory 116, and the processor 114 may be configured to execute computer executable instructions stored therein. The processor 114 may send or receive medical images 146 to the memory 116. For example, the processor 114 may receive medical images 146 from the image acquisition device 132 via the communication interface 118 and network 120 to be stored in memory 116. The processor 114 may also send medical images 146 stored in memory 116 via the communication interface 118 to the network 120 be stored in the database 124 or the hospital database 126.

The processor 114 may include a training module 151 that can train a deep learning (DL) network model using a set of training data, and generate the trained DL model 148 to predict a refined dose profile at a low statistical uncertainty (e.g., 1%). The training dataset may be constructed using dose data from prior simulations on a patient population. The simulations can be performed on the same or similar treatment area having a specific field size (e.g., same organ or tissue), optionally further on at least one treatment exclusion area. In an example, the training data may include a first set of dose profiles at one or more relatively high statistical uncertainty levels, and a second set of dose profiles at an uncertainty level lower than the first statistical uncertainty level. The first and second sets of dose profiles are both obtained from prior simulations on the same or similar treatment area. The first high-uncertainty dose profiles and the second low-uncertainty dose profiles each may be determined using an MC algorithm or a CCC algorithm. To train the DL model, the training module 151 can algorithmically adjust one or more DL model parameters (e.g., network layer node weights) until a specific training convergence criterion or a training stop criterion is met. The trained DL model 148 represents an established mapping or correspondence from the high-uncertainty dose profiles to the low-uncertainty dose profiles for the treatment area of a specific field size.

In some examples, multiple DL models may each be trained to predict a low-uncertainty dose profile for respective different treatment areas with respective field sizes. For example, a first DL model may be trained to map high-uncertainty dose profiles to low-uncertainty dose profiles for a first treatment area, a second DL model is trained to map high-uncertainty dose profiles to low-uncertainty dose profiles for a second treatment area different from the first treatment area, and so forth. Such multiple trained DL models, each corresponding to a specific treatment area, are referred to as treatment area-indexed DL models. In some examples, multiple DL models may each be trained to map respective high-uncertainty dose profiles at different uncertainty levels to a low-uncertainty level for the same treatment area. For example, a first DL model may be trained to map dose profiles at a first high-uncertainty level (e.g., 30%) to low-uncertainty dose profiles at a low-uncertainty level (e.g., 1%), a second DL model may be trained to map dose profiles at a different second high-uncertainty level (e.g., 20%) to the low-uncertainty dose profiles at the low-uncertainty level (e.g., 1%), and so forth. Such multiple trained DL models, each corresponding to a specific high-uncertainty level, are referred to as uncertainty level-indexed DL models.

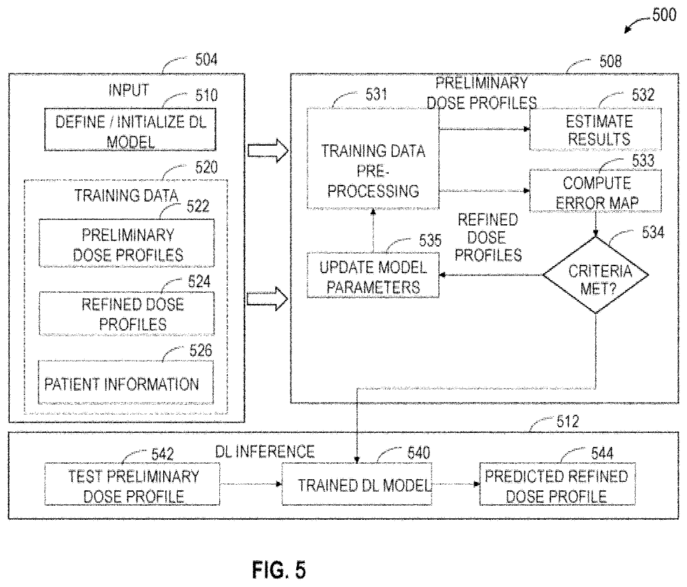

The trained DL model 148 can be stored in the memory 116. The training module 151 and the trained DL model 148 can alternatively be archived in a server, and accessed by one or more clients (e.g., TPS systems). Examples of training a DL model that maps a high-uncertainty dose profile to a low-uncertainty dose profile are discussed below with reference to FIG. 5.

The processor 114 may include a dose engine 152 that can calculate a dose profile (e.g., dose metrics, characteristics, distributions, or dose images, in various data formats) using the beam model 147. By way of example and not limitation, a Monte Carlo algorithm or a Collapsed Cone Convolution (CCC) algorithm may be used for dose calculation. Such dosimetry algorithms may be implemented as a software package stored in the software programs 144. Examples of the dose engine may include a voxel Monte Carlo (VMC) dose engine, an X-ray voxel Monte Carlo (XVMC) dose engine, or a GPU Monte Carlo Dose (GPUMCD).

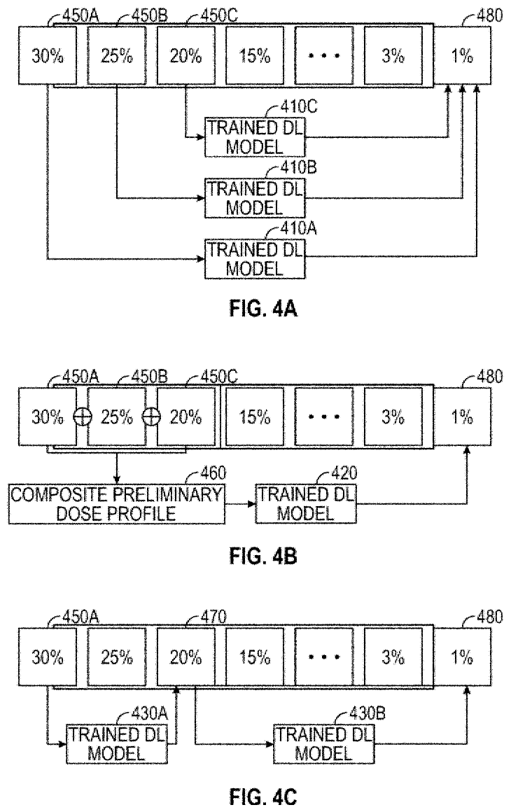

The dose engine 152 may calculate a preliminary high-uncertainty dose profile at a specific treatment area of a specific field size, apply said preliminary high-uncertainty dose profile to the trained DL model 148 to generate a prediction of a refined low-uncertainty dose profile for that treatment area. In a case where multiple DL models (e.g., the treatment area-indexed DL models or the uncertainty level-indexed DL models) each have been trained and stored in the memory 116 or archived in the server, the dose engine 152 may select a proper stored DL model according to the treatment area and/or the uncertainty level associated with the preliminary dose profile, apply said DL model to the preliminary dose profile to produce a prediction of a refined, low-uncertainty dose profile. In certain examples, the dose engine 152 may pre-process the preliminary high-uncertainty dose profile before the prediction process. Such pre-processing may include, for example, down-sampling, data truncation, or data filtering. The pre-processed preliminary high-uncertainty dose profile can then be fed into a trained DL model to generate a predicted low-uncertainty dose profile. In certain examples, the dose engine 152 may post-process the predicted refined low-uncertainty dose profile. Examples of such post-processing may include up-sampling, data interpolation or extrapolation, or data filtering. In some examples, the pre-processing or the post-processing operation can be incorporated into the trained DL model. Examples of predicting a refined, low-uncertainty dose profile using at least one trained DL model from one or more preliminary high-uncertainty dose profiles are described further below with reference to FIGS. 4A-4C.

The processor 114 may include at least a portion of a treatment planning system (TPS) configured to execute a treatment planning software (as part of the software programs 144), and generate the radiotherapy treatment plan 142 using the beam model 147, the medical images 146, and patient data 145. The medical images 146 may include information such as imaging data associated with a patient anatomical region, organ, or volume of interest segmentation data. In an example, the imaging data may include patient anatomical data corresponding to a mapping of at least a radiotherapy treatment area, optionally further of a treatment exclusion area. The patient data 145 may include information such as: functional organ modeling data (e.g., serial versus parallel organs, appropriate dose response models, etc.); radiation dosage data (e.g., DVH information); particle (e.g., electron) density; or other clinical information about the patient and treatment (e.g., other surgeries, chemotherapy, previous radiotherapy, etc.). The patient data 145 may include planning target volume (PTV) structures that enclose the clinical target volume (CTV) with anisotropic margins such as to account for possible uncertainties in beam alignment, patient positioning, organ motion, and organ deformation, and therefore ensure adequate treatment of the CTV.

The processor 114 can generate a beam model 147 for a particular radiation machine. In an example, to generate the beam model 147, the processor 114 can retrieve from multiple DL models stored archived the server, a trained DL model according to the collimator type and/or energy level associated with the radiation machine. The processor 114 can then apply machine scanning data (e.g., dose curves and dose statistics) acquired from the radiation machine to the retrieved trained DL model, which can produce as output values of a beam model parameters of the beam model 147.

The beam model 147 can be stored in the software programs 144. In an example, the beam model 147 may be presented to a user, such as being displayed on the display device 134. Other information may be presented to the user (e.g., displayed on the display device 134), such as a report containing beam model parameters, geometry information, dose calculation settings, and fitting results that show both measured dose distribution and calculated dose distribution based on the beam model. The fitting results. In an example, the beam model 147 may be delivered to a TPS for clinical treatment planning.

In some examples, the processor 114 may utilize software programs 144 to generate intermediate data such as updated parameters to be used, for example, by a machine learning model, such as a neural network model; or generate intermediate 2D or 3D images, which may then subsequently be stored in memory 116. The processor 114 may subsequently then transmit the executable radiotherapy treatment plan 142 via the communication interface 118 to the network 120 to the radiation therapy device 130, where the radiation therapy plan may be used to treat a patient with radiation. In addition, the processor 114 may execute software programs 144 to implement functions such as image conversion, image segmentation, deep learning, neural networks, and artificial intelligence. For instance, the processor 114 may execute software programs 144 that train or contour a medical image; such software programs 144 when executed may train a boundary detector or utilize a shape dictionary.

The processor 114 may be a processing device, include one or more general-purpose processing devices such as a microprocessor, a central processing unit (CPU), a graphics processing unit (GPU), an accelerated processing unit (APU), or the like. More particularly, the processor 114 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processor 114 may also be implemented by one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some embodiments, the processor 114 may be a special-purpose processor, rather than a general-purpose processor. The processor 114 may include one or more known processing devices, such as a microprocessor from the Pentium™, Core™, Xeon™, or Itanium family manufactured by Intel™, the Turion™, Athlon™, Sempron™, Opteron™ FX™, Phenom™ family manufactured by AMD™, or any of various processors manufactured by Sun Microsystems. The processor 114 may also include graphical processing units such as a GPU from the GeForce®, Quadro®, Tesla® family manufactured by Nvidia™, GMA, Iris™ family manufactured by Intel™, or the Radeon™ family manufactured by AMD™. The processor 114 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor (for example, a multi-core design or a plurality of processors each having a multi-core design). The processor 114 can execute sequences of computer program instructions, stored in memory 116, to perform various operations, processes, methods that will be explained in greater detail below.

In some examples, various functions performed by the processor 114, such as training of a DL model by the training module 151, generating and validation a beam model, and calculating or predicting dose using the dose engine 152, can be distributed in two or more processors, or in a client-server architecture. In an example, the training module 151 can be implemented in a server, and the dose engine 152 can be implemented in a client (e.g., a local TPS system). In an example, the trained DL model 148 can be archived in the server.

The memory 116 can store medical images 146. In some embodiments, the medical images 146 may include one or more MR images (e.g., 2D MRI, 3D MRI, 2D streaming MRI, four-dimensional (4D) MRI, 4D volumetric MRI, 4D cine MRI, etc.), functional MR images (e.g., fMRI, DCE-MRI, diffusion MRI), CT images (e.g., 2D CT, cone beam CT, 3D CT, 4D CT), ultrasound images (e.g., 2D ultrasound, 3D ultrasound, 4D ultrasound), one or more projection images representing views of an anatomy depicted in the MRI, synthetic CT (pseudo-CT), and/or CT images at different angles of a gantry relative to a patient axis, PET images, X-ray images, fluoroscopic images, radiotherapy portal images, SPECT images, computer generated synthetic images (e.g., pseudo-CT images), aperture images, graphical aperture image representations of MLC leaf positions at different gantry angles, and the like. Further, the medical images 146 may also include medical image data, for instance, training images, and ground truth images, contoured images, and dose images. In an embodiment, the medical images 146 may be received from the image acquisition device 132. Accordingly, image acquisition device 132 may include an MRI imaging device, a CT imaging device, a PET imaging device, an ultrasound imaging device, a fluoroscopic device, a SPECT imaging device, an integrated linac and MRI imaging device, or other medical imaging devices for obtaining the medical images of the patient. The medical images 146 may be received and stored in any type of data or any type of format that the data processing device 112 may use to perform operations consistent with the disclosed embodiments.

The memory 116 may be a non-transitory computer-readable medium, such as a read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a CD-ROM, a DVD or other optical storage, a cassette tape, other magnetic storage device, or any other non-transitory medium that may be used to store information including image, data, or computer executable instructions (e.g., stored in any format) capable of being accessed by the processor 114, or any other type of computer device. The computer program instructions can be accessed by the processor 114, read from the ROM, or any other suitable memory location, and loaded into the RAM for execution by the processor 114. For example, the memory 116 may store one or more software applications. Software applications stored in the memory 116 may include, for example, an operating system 143 for common computer systems as well as for software-controlled devices. Further, the memory 116 may store an entire software application, or only a part of a software application, that are executable by the processor 114. For example, the memory 116 may store one or more radiotherapy treatment plans 142.

The data processing device 112 can communicate with the network 120 via the communication interface 118, which can be communicatively coupled to the processor 114 and the memory 116. The communication interface 118 may provide communication connections between the data processing device 112 and radiotherapy system 100 components (e.g., permitting the exchange of data with external devices). For instance, the communication interface 118 may in some embodiments have appropriate interfacing circuitry to connect to the user interface 136, which may be a hardware keyboard, a keypad, or a touch screen through which a user may input information into radiotherapy system 100.

Communication interface 118 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), and the like. Communication interface 118 may include one or more digital and/or analog communication devices that permit data processing device 112 to communicate with other machines and devices, such as remotely located components, via the network 120.

The network 120 may provide the functionality of a local area network (LAN), a wireless network, a cloud computing environment (e.g., software as a service, platform as a service, infrastructure as a service, etc.), a client-server, a wide area network (WAN), and the like. For example, network 120 may be a LAN or a WAN that may include other systems S1 (138), S2 (140), and S3(141). Systems S1, S2, and S3 may be identical to data processing device 112 or may be different systems. In some embodiments, one or more of systems in network 120 may form a distributed computing/simulation environment that collaboratively performs the embodiments described herein. In some embodiments, one or more systems S1, S2, and S3 may include a CT scanner that obtains CT images (e.g., medical images 146). In addition, network 120 may be connected to Internet 122 to communicate with servers and clients that reside remotely on the internet.

Therefore, network 120 can allow data transmission between the data processing device 112 and a number of various other systems and devices, such as the OIS 128, the radiation therapy device 130, and the image acquisition device 132. Further, data generated by the OIS 128 and/or the image acquisition device 132 may be stored in the memory 116, the database 124, and/or the hospital database 126. The data may be transmitted/received via network 120, through communication interface 118 in order to be accessed by the processor 114, as required.

The data processing device 112 may communicate with the database 124 through network 120 to send/receive a plurality of various types of data stored on database 124. For example, the database 124 may store machine data associated with a radiation therapy device 130, image acquisition device 132, or other machines relevant to radiotherapy. The machine data information may include control points, such as radiation beam size, arc placement, beam on and off time duration, machine parameters, segments, MLC configuration, gantry speed, MRI pulse sequence, and the like. In an example, the database 124 may store training data used for training a DL model, such as one being trained to predict a refined, low-uncertainty dose profile from one or more preliminary high-uncertainty dose profiles. The training data may include a first set of high-uncertainty dose profiles (as model input), and a second set of low-uncertainty dose profiles (as “desired output” of the model). The high-uncertainty dose profiles and the low-uncertainty dose profiles may each be calculated using an MC algorithm or a CCC algorithm. The database 124 may be a storage device and may be equipped with appropriate database administration software programs. One skilled in the art would appreciate that database 124 may include a plurality of devices located either in a central or a distributed manner.

In some embodiments, the database 124 may include a processor-readable storage medium (not shown). While the processor-readable storage medium in an embodiment may be a single medium, the term “processor-readable storage medium” should be taken to include a single medium or multiple media (e.g., a centralized or distributed database, and/or associated caches and servers) that store the one or more sets of computer executable instructions or data. The term “processor-readable storage medium” shall also be taken to include any medium that is capable of storing or encoding a set of instructions for execution by a processor and that cause the processor to perform any one or more of the methodologies of the present disclosure. The term “processor readable storage medium” shall accordingly be taken to include, but not be limited to, solid-state memories, optical and magnetic media. For example, the processor readable storage medium can be one or more volatile, non-transitory, or non-volatile tangible computer-readable media.

The processor 114 may communicate with the database 124 to read images into the memory 116, or store images from the memory 116 to the database 124. For example, the database 124 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) data, projection images, graphical aperture images, etc.) that the database 124 received from image acquisition device 132. Database 124 may store data to be used by the processor 114 when executing software program 144, or when creating radiotherapy treatment plans 142. The data processing device 112 may receive the imaging data, such as a medical image 146 (e.g., 2D MRI slice images, CT images, 2D Fluoroscopy images, X-ray images, 3DMR images, 4D MR images, projection images, graphical aperture images, etc.) either from the database 124, the radiation therapy device 130 (e.g., an MR-linac), and or the image acquisition device 132 to generate a treatment plan 142.

In an embodiment, the radiotherapy system 100 may include an image acquisition device 132 that can acquire medical images (e.g., MR images, 3D MRI, 2D streaming MRI, 4D volumetric MRI, CT images, cone-Beam CT, PET images, functional MR images (e.g., fMRI, DCE-MRI and diffusion MRI), X-ray images, fluoroscopic image, ultrasound images, radiotherapy portal images, SPECT images, and the like) of the patient. Image acquisition device 132 may, for example, be an MRI imaging device, a CT imaging device, a PET imaging device, an ultrasound device, a fluoroscopic device, a SPECT imaging device, or any other suitable medical imaging device for obtaining one or more medical images of the patient. Images acquired by the image acquisition device 132 can be stored within database 124 as either imaging data and/or test data. By way of example, the images acquired by the image acquisition device 132 can be also stored by the data processing device 112, as medical image 146 in memory 116.

In an embodiment, for example, the image acquisition device 132 may be integrated with the radiation therapy device 130 as a single apparatus. For example, a MR imaging device can be combined with a linear accelerator to form a system referred to as an “MR-linac.” Such an MR-linac may be used, for example, to determine a location of a target organ or a target tumor in the patient, so as to direct radiation therapy accurately according to the radiotherapy treatment plan 142 to a predetermined target.

The image acquisition device 132 can be configured to acquire one or more images of the patient's anatomy for a region of interest (e.g., a target organ, a target tumor, or both). Each image, typically a 2D image or slice, may include one or more parameters (e.g., a 2D slice thickness, an orientation, and a location, etc.). In an embodiment, the image acquisition device 132 can acquire a 2D slice in any orientation. For example, an orientation of the 2D slice may include a sagittal orientation, a coronal orientation, or an axial orientation. The processor 114 can adjust one or more parameters, such as the thickness and/or orientation of the 2D slice, to include the target organ and/or target tumor. In an embodiment, 2D slices can be determined from information such as a 3D MRI volume. Such 2D slices can be acquired by the image acquisition device 132 in “real-time” while a patient is undergoing radiotherapy treatment, for example, when using the radiation therapy device 130, with “real-time” meaning acquiring the data in at least milliseconds or less.

The data processing device 112 may generate and store radiotherapy treatment plans 142 for one or more patients. The radiotherapy treatment plans 142 may provide information about a particular radiation dose to be applied to each patient. The radiotherapy treatment plans 142 may also include other radiotherapy information, such as control points including beam angles, gantry angles, beam intensity, dose-histogram-volume (DVH) information, number of radiation beams used during therapy, dose per beam, and the like.

The processor 114 may generate the radiotherapy treatment plan 142 by using software programs 144 such as treatment planning software (e.g., Monaco®, manufactured by Elekta AB of Sweden). In order to generate the radiotherapy treatment plans 142, the processor 114 may communicate with the image acquisition device 132 (e.g., a CT device, an MRI device, a PET device, an X-ray device, an ultrasound device, etc.) to access images of the patient and to delineate a target, such as a tumor. In some embodiments, the delineation of one or more OARs, such as healthy tissue surrounding the tumor or in close proximity to the tumor may be required. Therefore, segmentation of the OAR may be performed when the OAR is close to the target tumor. In addition, if the target tumor is close to the OAR (e.g., prostate in near proximity to the bladder and rectum), then by segmenting the OAR from the tumor, the radiotherapy system 100 may study the dose distribution not only in the target but also in the OAR.

In order to delineate a target organ or a target tumor from the OAR, medical images, such as MR images, CT images, PET images, fMR images, X-ray images, ultrasound images, radiotherapy portal images, SPECT images, and the like, of the patient undergoing radiotherapy may be obtained non-invasively by the image acquisition device 132 to reveal the internal structure of a body part. Based on the information from the medical images, a 3D structure of the relevant anatomical portion may be obtained. In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible). Other parameters that may be considered include the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or health care worker using a program such as Monaco manufactured by Elekta AB of Sweden) or automatically (e.g., using a program such as the Atlas-based auto-segmentation software, ABAS™, manufactured by Elekta AB of Sweden). In certain embodiments, the 3D structure of a target tumor or an OAR may be generated automatically by the treatment planning software.

After the target tumor and the OAR(s) have been located and delineated, a dosimetrist, physician, or healthcare worker may determine a dose of radiation to be applied to the target tumor, as well as any maximum amounts of dose that may be received by the OAR proximate to the tumor (e.g., left and right parotid, optic nerves, eyes, lens, inner ears, spinal cord, brain stem, and the like). After the radiation dose is determined for each anatomical structure (e.g., target tumor, OAR), a process known as inverse planning may be performed to determine one or more treatment plan parameters that would achieve the desired radiation dose distribution. Examples of treatment plan parameters include volume delineation parameters (e.g., which define target volumes, contour sensitive structures, etc.), margins around the target tumor and OARs, beam angle selection, collimator settings, and beam-on times. During the inverse-planning process, the physician may define dose constraint parameters that set bounds on how much radiation an OAR may receive (e.g., defining full dose to the tumor target and zero dose to any OAR; defining 95% of dose to the target tumor; defining that the spinal cord, brain stem, and optic structures receive ≤45 Gy, ≤55 Gy and <54 Gy, respectively). The result of inverse planning may constitute a radiotherapy treatment plan 142 that may be stored in memory 116 or database 124. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the data processing device 112 can generate a tailored radiotherapy treatment plan 142 having these parameters in order for the radiation therapy device 130 to provide radiotherapy treatment to the patient.

In addition, the radiotherapy system 100 may include a display device 134 and a user interface 136. The display device 134 may include one or more display screens that display medical images, interface information, treatment planning parameters (e.g., projection images, graphical aperture images, contours, dosages, beam angles, etc.) treatment plans, a target, localizing a target and/or tracking a target, or any related information to the user. The user interface 136 may be a keyboard, a keypad, a touch screen or any type of device that a user may input information to radiotherapy system 100. Alternatively, the display device 134 and the user interface 136 may be integrated into a device such as a tablet computer.

Furthermore, any and all components of the radiotherapy system 100 may be implemented as a virtual machine (e.g., VMWare, Hyper-V, and the like). For instance, a virtual machine can be software that functions as hardware. Therefore, a virtual machine may include at least one or more virtual processors, one or more virtual memories, and one or more virtual communication interfaces that together function as hardware. For example, the data processing device 112, the OIS 128, the image acquisition device 132 could be implemented as a virtual machine. Given the processing power, memory, and computational capability available, the entire radiotherapy system 100 could be implemented as a virtual machine.

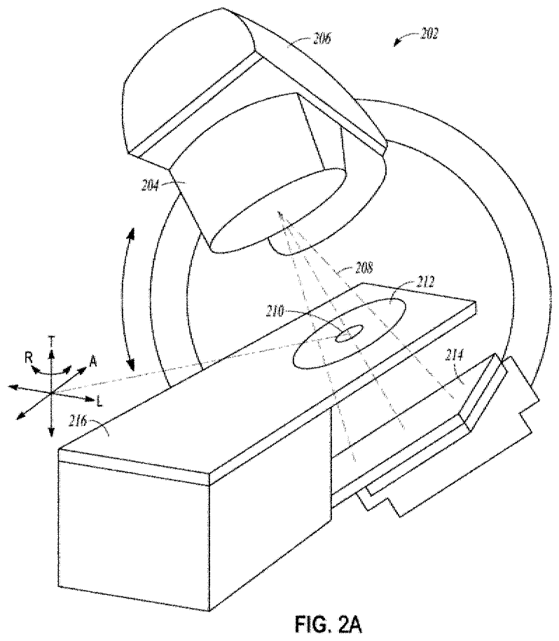

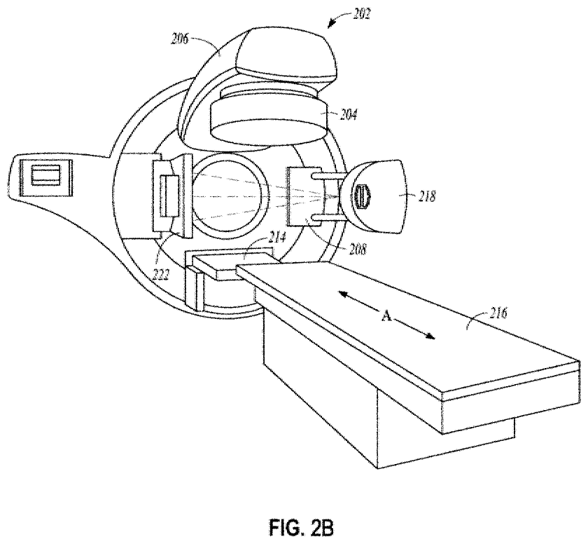

FIG. 2A illustrates an exemplary radiotherapy system 202 that may include a radiation source (e.g., an X-ray source or a linac), a couch 216, an imaging detector 214, and a radiation therapy output 204. The radiotherapy system 202 may be configured to emit a radiation beam 208 to provide therapy to a patient. The radiation therapy output 204 may include one or more attenuators or collimators, such as an MLC. A patient can be positioned in a region 212 and supported by the couch 216 to receive a radiation therapy dose, according to a radiotherapy treatment plan. The radiation therapy output 204 can be mounted or attached to a gantry 206 or other mechanical support. One or more chassis motors (not shown) may rotate the gantry 206 and the radiation therapy output 204 around the couch 216 when the couch 216 is inserted into the treatment area. In an embodiment, the gantry 206 may be continuously rotatable around the couch 216 when the couch 216 is inserted into the treatment area. In another embodiment, the gantry 206 may rotate to a predetermined position when the couch 216 is inserted into the treatment area. For example, the gantry 206 can be configured to rotate the therapy output 204 around an axis (“A”). The couch 216 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 216 movements or rotations in order to properly position the patient in or out of the radiation beam 208 according to a radiotherapy treatment plan. Both the couch 216 and the gantry 206 are independently moveable from one another in multiple degrees of freedom, which allows the patient to be positioned such that the radiation beam 208 can target the tumor. The MLC may be integrated with the gantry 206 to deliver the radiation beam 208 of a certain shape.

The coordinate system (including axes A, T, and L) shown in FIG. 2A can have an origin located at an isocenter 210. The isocenter can be defined as a location where the central axis of the radiation beam 208 intersects the origin of a coordinate axis, such as to deliver a prescribed radiation dose to a location on or within a patient. Alternatively, the isocenter 210 can be defined as a location where the central axis of the radiation beam 208 intersects the patient for various rotational positions of the radiation therapy output 204 as positioned by the gantry 206 around the axis A. As discussed herein, the gantry angle corresponds to the position of gantry 206 relative to axis A, although any other axis or combination of axes can be referenced and used to determine the gantry angle.

The linac system may have an imaging detector 214 that is preferably opposite the radiation therapy output 204. In an embodiment, the imaging detector 214 can be located within a field of the therapy beam 208. The imaging detector 214 can maintain alignment with the therapy beam 208. The imaging detector 214 can rotate about the rotational axis as the gantry 206 rotates. In an embodiment, the imaging detector 214 can be a flat panel detector (e.g., a direct detector or a scintillator detector). In this manner, the imaging detector 214 can monitor the therapy beam 208, or generate an image of the patient's anatomy. The control circuitry of radiotherapy system 202 may be integrated within system 100 or remote from it.

In an illustrative embodiment, one or more of the couch 216, the therapy output 204, or the gantry 206 can be automatically positioned, and the therapy output 204 can establish the therapy beam 208 according to a specified dose for a particular therapy delivery instance. A sequence of therapy deliveries can be specified according to a radiotherapy treatment plan, such as using one or more different orientations or locations of the gantry 206, the couch 216, or the therapy output 204. The therapy deliveries can occur sequentially, but can intersect in a desired therapy locus on or within the patient, such as at the isocenter 210. A prescribed dose of radiation therapy can thereby be delivered to the therapy locus while damage to tissue near the therapy locus can be reduced or avoided.

FIG. 2B illustrates an exemplary radiotherapy system 202 that combines a radiation system (e.g., a linac) and a CT imaging system. The radiation therapy output 204 may include an MLC (not shown). The CT imaging system may include an imaging X-ray source 218, such as providing X-ray energy in a kiloelectron-Volt (keV) energy range. The imaging X-ray source 218 can provide a fan-shaped and/or a conical beam 208 directed to an imaging detector 222, such as a flat panel detector. The radiotherapy system 202 can be similar to the system described in relation to FIG. 2A, such as including a radiation therapy output 204, a gantry 206, a couch 216, and another imaging detector 214 (such as a flat panel detector). The X-ray source 218 can provide a comparatively-lower-energy X-ray diagnostic beam, for imaging.

As illustrated in FIG. 2B, the radiation therapy output 204 and the X-ray source 218 can be mounted on the same rotating rotation mechanism, rotationally-separated from each other by 90 degrees. In some examples, two or more X-ray sources can be mounted along the circumference of the rotation mechanism, such that each has its own detector arrangement to provide multiple angles of diagnostic imaging concurrently. Similarly, multiple radiation therapy outputs 204 may be provided.

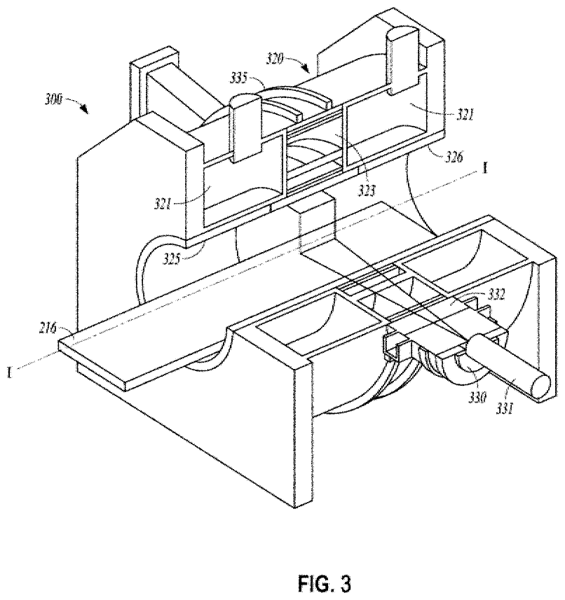

FIG. 3 illustrates an exemplary radiotherapy system 300 that combines a radiation system (e.g., a linac) and a nuclear MR imaging system, also referred to as an MR-linac system. The system 300 may include a couch 216, an image acquisition device 320, and a radiation delivery device 330. The system 300 can deliver radiation therapy to a patient in accordance with a radiotherapy treatment plan, such as the treatment plan 142 generated and stored in the memory 116. In some embodiments, the image acquisition device 320 may correspond to the image acquisition device 132 in FIG. 1 that may acquire images of a first modality (e.g., an MR image) or destination images of a second modality (e.g., a CT image).

The couch 216 may support a patient during a treatment session. In some implementations, the couch 216 may move along a horizontal translation axis (labelled “I”), such that the couch 216 can move the patient resting on the couch 216 into and/or out of the system 300. The couch 216 may also rotate around a central vertical axis of rotation, transverse to the translation axis. To allow such movement or rotation, the couch 216 may have motors (not shown) enabling movement of the couch 216. A controller (not shown) may control these movements or rotations in order to properly position the patient according to a treatment plan.

In some embodiments, the image acquisition device 320 may include an MR imaging machine that can acquire 2D or 3D MR images of the patient before, during, and/or after a treatment session. The image acquisition device 320 may include a magnet 321 for generating a primary magnetic field for magnetic resonance imaging. The magnetic field lines generated by operation of the magnet 321 may run substantially parallel to the central translation axis “I”. The magnet 321 may include one or more coils with an axis that runs parallel to the translation axis “I”. In some embodiments, the one or more coils in magnet 321 may be spaced such that a central window 323 of magnet 321 is free of coils. In other embodiments, the coils in magnet 321 may be thin enough or of a reduced density such that they are substantially transparent to radiation of the wavelength generated by radiotherapy device 330. In some embodiments, the image acquisition device 320 may also include one or more shielding coils, which may generate a magnetic field outside the magnet 321 of approximately equal magnitude and opposite polarity in order to cancel or reduce any magnetic field outside of the magnet 321. As described below, a radiation source 331 of radiotherapy device 330 may be positioned in the region where the magnetic field is cancelled, at least to a first order, or reduced.

The image acquisition device 320 may also include two gradient coils 325 and 326, which may generate a gradient magnetic field that is superposed on the primary magnetic field. The coils 325 and 326 may generate a gradient in the resultant magnetic field that allows spatial encoding of the protons so that their position can be determined. The gradient coils 325 and 326 may be positioned around a common central axis with the magnet 321 and may be displaced along that central axis. The displacement may create a gap, or window, between the coils 325 and 326. In embodiments where the magnet 321 includes a central window 323 between the coils, the two windows may be aligned with each other.

In some embodiments, the image acquisition device 320 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 320 concerns certain embodiments and is not intended to be limiting.

The radiotherapy device 330 may include the radiation source 331 (e.g., an X-ray source or a linac), and a collimator such as an MLC 332. A collimator is a beam-limiting device that can help to shape the beam of radiation emerging from the machine and can limit the maximum field size of a beam. The MLC 332 may be used for shaping, directing, or modulating an intensity of a radiation therapy beam to the specified target locus within the patient. The MLC 332 may include metal collimator plates, also known as MLC leaves, which slide into place to form the desired field shape. The radiotherapy device 330 may be mounted on a chassis 335. One or more chassis motors (not shown) may rotate chassis 335 around the couch 216 when the couch 216 is inserted into the treatment area. In an embodiment, chassis 335 may be continuously rotatable around the couch 216, when the couch 216 is inserted into the treatment area. The chassis 335 may also have an attached radiation detector (not shown), preferably located opposite to radiation source 331 and with the rotational axis of chassis 335 positioned between radiation source 331 and the detector. Further, device 330 may include control circuitry (not shown) used to control, for example, one or more of the couch 216, image acquisition device 320, and radiotherapy device 330. The control circuitry of radiotherapy device 330 may be integrated within system 300 or remote from it.

During a radiotherapy treatment session, a patient may be positioned on the couch 216. System 300 may then move the couch 216 into the treatment area defined by magnetic 321 and coils 325, 326, and chassis 335. Control circuitry may then control the radiation source 331, MLC 332, and the chassis motor(s) to deliver radiation to the patient through the window between coils 325 and 326 according to a radiotherapy treatment plan.

The radiation therapy output configurations illustrated in FIGS. 2A-2B and 3, such as the configurations where a radiation therapy output can be rotated around a central axis (e.g., an axis “A”), are for the purpose of illustration and not limitation. Other radiation therapy output configurations may be used. For example, a radiation therapy output can be mounted to a robotic arm or manipulator having multiple degrees of freedom. In yet another embodiment, the therapy output can be fixed, such as located in a region laterally separated from the patient, and a platform supporting the patient may be used to align a radiation therapy isocenter with a specified target locus within the patient.

FIGS. 4A-4C illustrate examples of AI-based dose prediction such as using the dose engine 152 of the radiotherapy system 100. The dose prediction involves predicting a refined low-uncertainty dose profile from one or more preliminary high-uncertainty dose profiles using one or more trained DL models, such as the DL model 148 generated by the training module 151. FIG. 4A illustrates DL models 410A, 410B, and 410C each having been trained to establish a mapping (or correspondence) from high-uncertainty dose profiles at respective uncertainty levels 30%, 25%, and 20%, to a low-uncertainty dose profile at a clinically acceptable uncertainty level 1%. The trained DL models 410A, 410B, and 410C can be stored in the memory 116 or archived in the server. During the radiotherapy treatment planning, to predict a refined dose profile at an uncertainty level of 1%, the dose engine 152 can first calculate a preliminary dose profile using a dose algorithm (e.g., MC algorithm or CCC algorithm) at a high-uncertainty level, such as one of the preliminary dose profiles 450A at 30% uncertainty level, 450B at 25% uncertainty level, or 450C at 20% uncertainty level. The dose engine 152 can then apply the preliminary high-uncertainty dose profile to an appropriate trained DL model, such as applying the preliminary dose profiles 450A to the trained DL model 410A, the preliminary dose profile 450B to the trained DL model 410B, or the preliminary dose profile 450C to the trained DL model 410C. The trained DL models 410A, 410B, or 410C can each produce a prediction of a refined dose profile 480 at a low-uncertainty level of 1%.

FIG. 4B illustrates a DL model 420 that has been trained (e.g., using the training module 151) to establish a mapping (or correspondence) from composite preliminary high-uncertainty dose profiles to a low-uncertainty dose profiles at a clinically acceptable low-uncertainty level (e.g., 1%). The composite preliminary high-uncertainty dose profiles can be generated by aggregating (e.g., concatenating) two or more preliminary high-uncertainty dose profiles with distinct uncertainty levels, such as 30%, 25%, and 20% in an example. The trained DL model 420 can be stored in the memory 116 or archived in the server. During the radiotherapy treatment planning, to predict a refined dose profile (e.g., at an uncertainty level of 1%), the dose engine 152 can first calculate a plurality of preliminary dose profiles using a dose algorithm (e.g., MC algorithm or CCC algorithm) at high-uncertainty levels, such as preliminary dose profiles 450A at 30% uncertainty level, 450B at 25% uncertainty level, and 450C at 20% uncertainty level. The dose engine 152 can then aggregate (e.g., concatenate) the preliminary dose profiles 450A, 450B, and 450C to form a composite preliminary dose profile 460, apply the composite preliminary dose profile 460 to the trained DL model 420 to produce a prediction of a refined dose profile 480 at a low-uncertainty level of 1%.

In addition or alternative to prediction of a low-uncertainty dose profile directly from one or more preliminary high-uncertainty dose profiles as described above with respect to FIGS. 4A and 4B, in some examples, the dose engine 152 may predict a low-uncertainty dose profile in multiple, sequential steps, where an intermediate dose prediction at an intermediate uncertainty level can be further used to predict a dose profile at a lower uncertainty level. FIG. 4C illustrates a first DL model 430A and a second DL model 430B, both of which can be trained using the training module 151. The first trained DL model 430A establishes a mapping (or correspondence) from a preliminary high-uncertainty dose profile at 30% uncertainty level to an intermediate dose prediction at 20% uncertainty level. The second trained DL model 430B establishes a mapping (or correspondence) from a preliminary high-uncertainty dose profile at 20% uncertainty level to a refined dose profile at a clinically acceptable uncertainty level of 1%. The trained DL models 430A and 430B can be stored in the memory 116 or archived in the server. During the radiotherapy treatment planning, to predict a refined dose profile (e.g., at the uncertainty level of 1%), the dose engine 152 can first calculate a preliminary dose profile 450A at a uncertainty level of 30%, and apply the preliminary dose profile 450A to the first trained DL model 430A to generate a predicted intermediate dose profile 470 at an uncertainty level of 20%. The dose engine 152 can then apply the predicted intermediate dose profile 470 to the second trained DL model 430B, which can produce a prediction of a refined dose profile 480 at a low-uncertainty level of 1%.

FIG. 5 illustrates an exemplary process 500 for training a deep learning (DL) model to predict a refined low-uncertainty dose profile using a pre-calculated preliminary, high-uncertainty dose profile. The process 500 can be implemented as computer-readable and executable instructions and executed by the training module 151. Input 504 may include a DL model 510 having an initial network architecture and initial parameter settings. Examples of the DL model 510 may include a convolutional neural network (CNN), a recurrent neural network (RNN), a deep belief network (DBN), or a hybrid neural network comprising two or more neural network models of different types or different model configurations. In some examples, the DL model 510 may include a CNN with a U-Net architecture for fast and precise segmentation of images. The U-Net architecture generally includes a contracting path and an expansive path, which gives it a “U”-shaped architecture. The contracting path is a typical convolutional network that consists of repeated application of convolutions, each followed by a rectified linear unit (ReLU) and a max pooling operation. During the contraction, the spatial information is reduced while feature information is increased. The expansive pathway combines the feature and spatial information through a sequence of up-convolutions and concatenations with high-resolution features from the contracting path. Examples of U-Net and its variants used for predicting a refined low-uncertainty dose profile from a preliminary high-uncertainty dose profile are described below with reference to FIGS. 6A-6D.

The input 504 may include training data 520 that may be used for training the DL model 510. As depicted, the training data 520 may include preliminary high-uncertainty dose profiles 522 and refined low-uncertainty dose profiles 524. The preliminary high-uncertainty dose profiles 522 are the input to the DL model, and the refined low-uncertainty dose profiles 524 are the “desired output” (or “target labels”) of the DL model being trained. The input high-uncertainty dose profiles 522 and the “desired output” low-uncertainty dose profiles 524 may each be calculated using dose data obtained from dose simulations. The dose engine 152 may use a dose calculation algorithm (e.g., MC algorithm or CCC algorithm) to calculate dose profiles at respective uncertainty levels. The dose profiles may be represented by dose metrics, characteristics, distributions, or dose image in various data formats.

In an example, the preliminary high-uncertainty dose profiles 522 and the refined low-uncertainty dose profiles 524 can each include respective dose images (or image data such as two- or three-dimensional dose matrices) representing spatial distribution of dose data across at least one treatment area and optionally at least one treatment exclusion area. In an example, the dose image can be a two-dimensional or three-dimensional image that comprises respective areas representing dose data using respective image color channels or respective values in an image channel. In another example, the dose image can be a two-dimensional grayscale image. In some examples, the radiotherapy dose image can be produced from linear interpolation or nearest neighbor interpolation of archived dose data at a lower resolution; or the radiotherapy dose data comprises an indication of an amount of radiotherapy treatment at the coordinates within the coordinate space of the anatomical area. In some examples, the preliminary dose profile and the refined dose profile each include at least one dose metric indicative of a statistical distribution of the dose. Examples of the dose metric may include a percentage depth dose (PDD) curve that characterizes relative dose quantity determined as the ratio between the axis dose at a specific depth and the axis dose at a reference dose depth; a dose profile (or a dose distribution) that characterizes off-axis dose distribution, such as doses at diagonals, a percentage radial dose (PRD) curve representing changes of relative dose with a radial distance; a dose-volume histogram, an overlap volume histogram, or a three-dimensional dose distribution.

In some examples, the training data 520 may additionally include patient information 526. Examples of the patient information 526 may include functional organ modeling data, particle (e.g., electron) density; planning target volume (PTV) structures that enclose the clinical target volume (CTV) with anisotropic margins such as to account for possible uncertainties in beam alignment, patient positioning, organ motion, and organ deformation, and therefore ensure adequate treatment of the CTV. The training data 520 may additionally include machine settings data including information about settings of the radiation machine.

The DL training process 508 can apply the training data 520 to train the DL model 510. In some examples, noise maybe added to the preliminary dose profiles 522 to more realistically represent scanning data in a clinical setting. For example, the difference between the calculated dose profile (e.g., from prior simulations) and the measured dose profile, referred to as dose noise, can be added to at least a portion of the preliminary dose profiles 522 to produce “noisy” training data. The noisy training data can improve the robustness of the trained DL model. In an example, the training data 520 may reside in a local client (e.g., a TPS system), and the DL training process 508 can be implemented in a server. The training data 520 may be compressed, and then transferred to the server via a communication channel.

The DL training process 508 may include a training data pre-processing 531 step to pre-process the training data. Examples of data pre-processing 531 may include, for example, down-sampling, data truncation, denoising, filtering, or de-nosing of the preliminary dose profiles 522. The training data may be converted into a desired data format, such as re-arranging sequences of PDD data and/or the dose profile data in the 2D dose matrix, without changing the value of data points for feature recognition. The training data pre-processing 531 may additionally or alternatively include operations to mitigate certain deficiencies of the training data, or to improve the efficiency of model training process. In an example, the data pre-processing 531 may include a denoising operation (e.g., a computational model) to remove or attenuate noise from the preliminary dose profiles 522.

In some examples, at least a portion of the data pre-processing 531 can be integrated into the DL model. For example, the denoising can be implemented as a fully connected layer of the DL model (e.g., a CNN model). The denoising layer in the CNN may be trained separately from the rest of the DL model. Alternatively, the denoising layer may be trained together with the rest of the DL model. The inclusion of the denoising layer can improve the dose predication performance of the trained DL model.

The pre-processed preliminary high-uncertainty dose profiles can then be fed into the DL model 510 to generate estimated results 532. The estimated results 532 can be compared to the refined low-uncertainty dose profiles 524 (the “desired output”). At 533, an estimation error can be computed such as a difference between the estimated results 532 and the refined low-uncertainty dose profiles 524. The estimation error can be compared to model convergence or training stop criteria at 534, such as proceeding to a sustained minimum for a specified number of training iterations. If the convergence or training stop criteria has not been satisfied, the estimation error may be used to update DL model parameters 535 (0, e.g., layer node weights and biases), such as through backpropagation, to reduce or minimize errors in the machine parameter or the estimations errors during subsequent training trials. Another batch of training data can then be selected from the training data 520 and expected results for another iteration of DL model training. In an embodiment, model parameter update using the estimation error may be carried out to minimize or reduce a loss function (or objective function, or cost function). An example of the loss function is square estimation error, as given in Equation (1):

$$J ( {Θ}^{*} ) = arg {min}_{Θ} {‖ Y - {Y}^{*} ‖}^{2} \qquad ( 1 )$$

where Y can represent feature values extracted from the estimated results 532, Y* can represent feature values extracted from the refined low-uncertainty dose profiles 524, and where * can represent ideal parameters of the DL model (e.g., layer node weights and biases as described above) that minimize the squared error between Y and Y*. Other loss functions may be used, such as log cos h (logarithm of the hyperbolic cosine of the prediction error), mean absolute error (MAE), mean squared error (MSE), weighted MSE, Huber loss (smooth MAE), or quantile loss, among others. The values of DL model parameters (Θ) can be iteratively improved.

After updating the parameters of the DL model, the iteration index can be incremented by one. The iteration index can correspond to the number of times that the parameters of the DL model have been updated. Convergence or training stop criteria can be checked at 534. In an embodiment, the convergence or stop criteria may include a value of the iteration index in comparison to a threshold number of iterations. In an embodiment, the convergence or stop criteria may include an estimation error, such as cumulative loss (e.g., squared error between Y and Y* in Equation (1) above) over multiple training trials, in comparison to an error threshold.

If at 534 it is determined that the convergence or stop criteria have been satisfied (e.g., the iteration index exceeding the threshold number of iterations, or the cumulative loss falling below the error threshold), then the training process can be stopped. The trained DL model 540 can be saved in the memory 116 of data processing device 112, or in a server. Additionally or alternatively, a report containing information about the trained DL model 540 can be output to a user via the user interface 136.

The trained DL model 540 can be validated using validation data. The validation data can be generated using a similar approach to the generation of training data 520. In an example, the validation data may include preliminary high-uncertainty dose profiles and refined low-uncertainty dose profiles of the same treatment area. The high-uncertainty dose profiles and the low-uncertainty dose profiles in the validation dataset may be different from those in the training data 520 used for DL training process 508. Applying the validation data to the trained DL model 540 can yield predicted low-uncertainty profiles. A performance metric can be evaluated. The trained DL model 540 is deemed to pass the validation check, and deployed to the testing or inference process 512 only when the performance metric satisfies a validation criterion. One example of the model performance metric is a dose difference metric, i.e., the difference between the measured dose distribution and the predicted dose distribution. Other examples of the model performance may include a radiotherapy planning target volume (PTV) dose coverage metric, a gamma passing rate (the percentage of measurement points satisfying the condition of gamma index less than 1, where the gamma index is a dimensionless metric representing combined dose difference and distance difference between the the measured dose distribution and the predicted dose distribution), or structure similarity.

Once validated, the trained DL model 540 can be deployed to a testing or inference process 512 for dose prediction. A test preliminary dose profile 542 may be calculated using a dose calculation algorithm (e.g., MC algorithm or CCC algorithm) at a high-uncertainty level. The test preliminary dose profile 542 is then fed into the trained DL model 540, and a prediction of a refined low-uncertainty dose profile 544 may be determined. The TPS system can use such predicted refined dose profile to generate a radiotherapy treatment plan, or to update an existing radiotherapy treatment plan. In some examples, an initial dose radiotherapy treatment plan may be used to calculate the preliminary dose profile (such as by the dose engine 152). The refined dose profile predicted from the preliminary dose profile can then be used to update the initial radiotherapy treatment plan in a process of treatment plan optimization. In some examples, the radiotherapy treatment plan thus generated can be compared against a previously computed dose (such as stored in an existing radiotherapy treatment plan). The comparison result can be provided to a user. In some examples, the radiotherapy treatment plan thus generated can be examined and tuned by a human modeler (e.g., a modeling physicist). A radiotherapy machine such as the radiation therapy device 130 can generate a radiotherapy for delivery to the patient in accordance with the radiotherapy treatment plan.

FIGS. 6A-6D illustrate examples of CNN models with a U-Net architecture or a variant thereof that are trained to predict a refined low-uncertainty dose profile 604 (as an output of the model) using a preliminary high-uncertainty dose profile 602 (as an input to the model). The trained CNN models with the U-Net architecture(s) can be embodiments of the DL model 148 in FIG. 1, the trained DL model 540 in FIG. 5, or any of the trained DL models 410A-410C, 420, and 430A-430B.

FIG. 6A illustrates a standard U-Net architecture 610 adapted for generating an output data set, such as a refined, low-uncertainty dose profile (e.g., a dose image) based on an input training set, which may include one or more preliminary, high-uncertainty dose profiles (e.g., one or more dose images). In particular, the U-Net architecture 610 may be used for semantic segmentation of dose images. As illustrated in FIG. 6A, the U-Net architecture 610 consists of a contracting path 612 (left branch of the U-Net, also referred to as an encoding operation) that learns a set of dose image features, and an expansive path 614 (right branch of the U-Net, also referred to as a decoding operation) that reconstructs the output dose image. The contracting path 612 includes operations as used in a typical CNN architecture, which consists of multiple layers of repeated application of two 3-by-3 convolutions (unpadded convolutions) each followed by a rectified linear unit (ReLU) (jointly referred to as “Conv/ReLU”, denoted by the right-pointing arrow 615A), and a 4-by-4 max pooling operation with stride 2 for down-sampling (denoted by the down-pointing arrow 615B). At each down-sampling step, the number of feature channels can be doubled. Proceeding down the contracting path 612 from one layer to another, the size of the features by convention increases by a factor of 2.

The expansive path 614 consists of multiple layers of repeated application of up-sampling of the feature map, followed by a 4-by-4 de-convolution (or up-convolution) that halves the number of feature channels, followed by a ReLU (jointly referred to as “DeConv/ReLU”, denoted by the up-pointing arrow 615C), a concatenation with the correspondingly cropped feature map from the contracting path 612 of the same level (also referred to as “copy/cropping” or “skip connection”, denoted by the right-pointing arrow 615E), and two 3-by-3 convolutions each followed by a ReLU (jointly referred to as “Conv/ReLU”, denoted by the right-pointing arrow 615A). The copy/cropping step (as indicated by 615E) is added to mitigate the loss of border pixels in every convolution. The final layer of the U-Net architecture 610 includes a 1-by-1 convolution that maps each 64-component feature vector to the desired number of classes, followed by a sigmoid activation function (jointly referred to as “Conv/Sigmoid”, denoted by the right-pointing arrow 615D).

When the trained U-Net architecture 610 is used inference or testing, the input would be a preliminary, high-uncertainty dose profile (e.g., one or more dose images), and the output would be a prediction of a refined, low-uncertainty dose profile (e.g., a dose image).

FIG. 6B illustrates a Hierarchically Densely connected U-Net (HD U-Net) 620. The Dense U-Net, which is based on the standard U-net structure, employed the dense concatenation to deepen the depth of the network architecture and achieve feature reuse. The HD U-Net 620 adds in the densely connected convolutional layers 625A into the U-Net architecture, along with densely connected down-sampling blocks 625B, and a U-Net up-sampling blocks 625C. The HD U-Net was trained to take as input the preliminary high-uncertainty dose profile 602, and to predict the refined low-uncertainty dose profile 604.

FIG. 6C illustrates an Attention U-Net 630, a variant of the standard U-Net with additive attention gates 635. In the standard U-Net as illustrated in FIG. 6A, during up-sampling in the expanding path 614, spatial information recreated may be imprecise. To counteract this problem, the U-Net uses skip connections that combine spatial information from the down-sampling in the expanding path 614 together with the up-sampling in the contracting path 612. However, this brings across many redundant low-level feature extractions, as feature representation is poor in the initial layers. The attention gates 635 are used to enhance activation to only the relevant portions of the image, and suppress the redundant or irrelevant portions of the image during training. This reduces the computational resources wasted on irrelevant activations, providing the network with better generalization power.

The additive attention gates 635 implemented in the attention U-Net 630 can be additive soft attention gates. Unlike hard attention which takes a binary decision on whether to keep or to crop a given region of an image, soft attention applies weights to different portions of the image depending on their respective relevance. Areas of high relevance is multiplied by larger weights and areas of low relevance is multiplied by smaller weights, such that “more attention” is given the relevant regions with higher. During the model training, the weights can also be trained to determine optimal weights for different regions of the image, such that the attention U-Net can actively suppress activations (i.e., reducing weights) in irrelevant regions, reducing the number of redundant features.

FIG. 6D illustrates a schematic of the additive soft attention gate 635, such that used in the attention U-Net 630. An AG can take in two inputs, vectors x and g. The vector g is taken from the next lowest layer of the network. As vector g comes from deeper into the network, it has smaller dimensions (F)×height (H)×width (W) than vector x and better feature representation. In an example, vector x has a dimension of 64×64×64, and the g vector has a dimension of 32×32×32. The vector x goes through a strided convolution, and vector g goes through a 1×1 convolution. The two vectors x and g are then summed element-wise. Then aligned weights become larger, while unaligned weights become relatively smaller. The resultant vector goes through a ReLU activation layer and a lxi convolution to collapse the dimension to, e.g., 1×32×32. This vector then goes through a sigmoid layer which scales the vector between 0 and 1, producing the attention coefficients (weights), where coefficients closer to 1 indicate more relevant features. The attention coefficients can then be up-sampled to the original dimensions (e.g., 64×64) of the x vector using trilinear interpolation. The attention coefficients are multiplied element-wise to the original x vector, scaling the vector according to relevance. This is then passed along in the skip connection as normal.

FIG. 6E illustrates a Residual U-Net 650 with convolutional encoding and decoding units using recurrent convolutional layers (RCLs), which is based on a standard U-Net architecture. The standard U-Net architecture as shown in 610A uses a forward convolutional unit “Conv/ReLU” that comprising two convolutions each followed by a ReLU. Different variations of the standard U-Net can be designed with variations of this forward convolutional unit. In an example, recurrent convolutional units 655A may be used in the contracting path 612, and recurrent up-convolution units 655B may be used in the expanding path 614. Such U-Net with the recurrent convolutional units and recurrent up-convolution units is also referred to as recurrent U-Net, or RU-Net. In another example, forward convolutional layers with residual connectivity may be used in the contracting path 612 and the expanding path 614. Such U-Net with the residual connectivity is also referred to as residual U-Net, or ResU-Net. In yet another example, recurrent convolutional layers with residual connectivity may be used in the contracting path 612 and the expanding path 614. Such U-Net with the recurrent convolutional layer and residual connectivity is also known as R2U-Net. Combining the standard U-Net with the recurrent convolutional units, residual units, or recurrent convolutional units along with residual connectivity can offer a number of benefits. For example, a residual unit helps when training deep architectures. Feature accumulation with recurrent residual convolutional layers ensures better feature representation for segmentation tasks. The U-Net architectures with various extended features as described above can improve the performance of image segmentation and accuracy of dose profile prediction, yet without substantially increasing the model complexity (e.g., the number of network parameters) or training time.

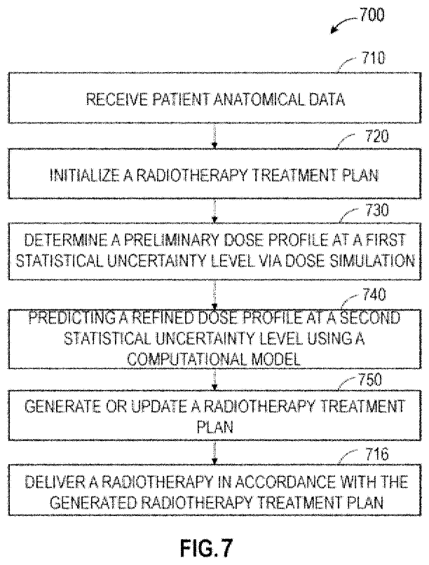

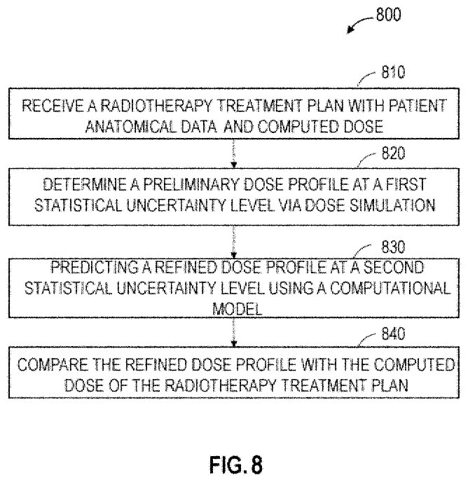

FIGS. 7-8 illustrate exemplary methods 700 and 800 of predicting a radiation dose profile using artificial intelligence (AI)-based techniques, and using the predicted dose profile in a radiotherapy treatment planning process or a secondary dose check process. A refined dose profile at a low statistical uncertainty level can be predicted using a pre-calculated preliminary dose profile using a computational model, such as a trained deep learning (DL) network model. The refined dose profiled can then be used to generate a radiotherapy treatment plan, or to check against a computed dose profile of a radiotherapy treatment plan. One or more of the methods 700 or 800 may be implemented in and executed by the radiotherapy system 100, which may include a radiation device to provide radiotherapy to a subject according to a treatment plan.

FIG. 7 illustrates a method 700 of predicting a radiation dose profile and planning a radiotherapy treatment using the predicted dose profile. The method 700A begins at step 710, where patient data anatomical data can be received and used for initial dose calculation. The patient anatomical data corresponds to a mapping of at least a radiotherapy treatment area, optionally further of a treatment exclusion area. Other data from the patient data 145 and/or medical images 146 as described above with respect to FIG. 1 can also be received.

At step 720, a radiotherapy treatment plan can be initialized using the patient anatomical data received at step 711, such as using a treatment planning system (TPS) implemented in the processor 114 of the radiotherapy system 100. In some examples, the radiotherapy treatment plan can be examined and tuned by a human modeler (e.g., a modeling physicist) until it is ready to be deployed to the TPS.

At step 730, a preliminary dose profile can be determined using the radiotherapy treatment plan generated at step 720 (or the updated radiotherapy treatment plan from 750, as discussed further below). The preliminary dose profile can be calculated using the dose engine 152 in a dose simulation process using a dose calculation algorithm which, by way of example and not limitation, may include a Monte Carlo (MC) algorithm or a Collapsed Cone Convolution (CCC) algorithm. The preliminary dose profile may be represented by a dose distribution image (or simply referred to as a “dose image”). A dose image can take the form of a two-dimensional (2D) or three-dimensional (3D) dose matrix representing a spatial distribution of dose data across at least one treatment area and optionally at least one treatment exclusion area.

The preliminary dose profile can be calculated at a first statistical uncertainty level of dose calculation. As described above, the number of particles (also referred to as histories) involved in dose simulation are related to the statistical uncertainty of final dose distribution. The uncertainty of dose calculation reflects the accuracy and robustness of dose calculation. As the number of histories (i.e., simulated particles per voxel) is increased, the statistical dose uncertainties decreases. However, the calculation of a low-uncertainty dose profile using the MC or CCC algorithms generally requires intensive computation and may take a significant amount of computation time. Various embodiments as described in this document, such as the methods 700A and 700B, can predict a dose profile at sufficiently low and clinically acceptable statistical uncertainties yet with substantially reduced computational time, thereby improving the dose calculation efficiency and accuracy.

The trained DL model can be validated before being used in predicting dose profile (the testing or inference process 512 as shown in FIG. 5). Validation data can be generated using a similar approach to the generation of training data. A performance metric can be evaluated from model validation. The trained DL model is deemed to pass the validation check, and deployed to the testing or inference process 512 only when the performance metric satisfies a validation criterion. Examples of the model performance may include one or more of a dose difference metric, a radiotherapy planning target volume (PTV) dose coverage metric, a gamma passing ratio, or a structure similarity.

At step 740, a refined dose profile can be predicted from the preliminary dose profile produced at step 730. The refined dose profile has at a second statistical uncertainty level lower than the first statistical uncertainty level of the preliminary dose profile. The prediction may include applying the determined preliminary dose profile to a computational model. In an example, the computational model can be a trained deep learning (DL) network model, such as one of the DL model 148 shown in FIG. 1, the trained DL models 410A-410C, 420, or 430A-430B shown in FIGS. 4A-4C, or the trained DL model 540 shown in FIG. 5. Examples of the DL model may include a convolutional neural network (CNN), a recurrent neural network (RNN), a long-term and short-term memory (LSTM) network, a deep belief network (DBN), or a hybrid neural network comprising two or more neural network models of different types or different model configurations. The architectures and parameters of the DL model can vary. In some examples, the DL model being used at 740 for predicting a refined dose profile can include a CNN model with a U-Net architecture or a variant thereof, such as a standard U-Net, a Hierarchy Dense (HD) U-Net, an Attention U-Net, a Group Normalization (GN) U-Net, or a Recurrent Residual U-Net, as describe above with reference to FIGS. 6A-6E.

The DL model can be trained using the training module 151. As discussed above, training of the DL model can be carried out using the exemplary process 500. As described above, the DL model training may include constructing training data obtained from prior dose simulations on a radiotherapy treatment area, where the training data may comprise a first set of dose profiles at the first statistical uncertainty level, and a second set of dose profiles at the second statistical uncertainty level. The training data can be fed into a DL model, such that the first set of dose profiles are used as model input, and the second set of dose profiles are used as “desired output” of the model being trained. During the training, the model parameters can be adjusted until convergence or stop criteria have been satisfied. The trained DL model represents an established mapping (or correspondence) from the first set of dose profiles (of higher uncertainty levels) to the second set of dose profiles (of lower-uncertainty levels) for the same radiotherapy treatment area.

In some examples, a number of distinct DL models may be separately trained to establish respective mappings from high-uncertainty dose profile at respective different uncertainty levels (e.g., 30%, 25%, and 20%) to a low-uncertainty dose profile at a clinically acceptable uncertainty level (e.g., 1%). During the radiotherapy treatment planning, depending on the preliminary dose profiled and its uncertainty level, a proper trained DL model can be used to predict a refined dose profile, as depicted in FIG. 4A.

In some examples, the training data being used for training a DL model may comprise two or more preliminary high-uncertainty dose profiles at distinct uncertainty levels, all obtained from the prior dose simulations on the same radiotherapy treatment area. The two or more preliminary high-uncertainty dose profiles may be aggregated (e.g., concatenated) to form composite preliminary high-uncertainty dose profiles, which can be used as input to the model during model training. The trained DL model establishes a mapping (or correspondence) from the composite dose profiles of higher uncertainty levels to the second set of dose profiles of a lower uncertainty level. During the radiotherapy treatment planning, dose stimulations can be performed to determine a first preliminary dose profile at the first statistical uncertainty level and a second preliminary dose profile at the third statistical uncertainty level. The first preliminary dose profile can be aggregated with the second preliminary dose profile to form a composite dose profile, which can be fed into the trained DL model to generate a prediction of a refined dose profile at a lower uncertainty level, as depicted in in FIG. 4B.

In some examples, the prediction of dose profile can be carried out in multiple, sequential steps, where an intermediate dose prediction at an intermediate uncertainty level can be further used to predict a dose profile at a lower uncertainty level. The training data being used for training a DL model may comprise an intermediate set of dose profiles obtained from the prior dose simulations on the radiotherapy treatment area at an intermediate statistical uncertainty level lower than the first statistical uncertainty level and higher than the second statistical uncertainty level. The model training can include training a first DL model to establish a mapping (or correspondence) from the first set of dose profiles to the intermediate set of dose profiles, and training a second DL model to establish a mapping (or correspondence) from the intermediate set of dose profiles to the second set of dose profiles. During the radiotherapy treatment planning, a preliminary high-uncertainty dose profile can be applied to the first trained DL model to predict an intermediate dose profile at the intermediate statistical uncertainty level. The predicted intermediate dose profile can then be applied to the second trained DL model to predict the refined low-uncertainty dose profile, as depicted in FIG. 4C.

At 750, a radiotherapy treatment plan can be generated or updated based at least in part on the refined dose profile. In an example, the exiting radiotherapy treatment plan initialized at 720 can be updated based on the predicted refined dose profile at step 740 and received patient anatomical data 710 if the radiotherapy treatment plan satisfies a specific condition, or if the predicted refined dose profile satisfies a dose criterion to be delivered to the radiotherapy treatment plan. In some examples, if the predicted refined dose profile does not satisfy the dose criterion, the updated radiotherapy treatment plan can be used to compute preliminary dose profile at 730, and a new refined dose profile can be predicted at 740.

At 760, a radiotherapy machine such as the radiation therapy device 130 can generate radiotherapy for delivery to the patient in accordance with the generated or updated radiotherapy treatment plan.

FIG. 8 is a flow chart illustrating another exemplary method predicting a radiation dose profile and planning a radiotherapy treatment using the predicted dose profile. The method 800 can predict a refined dose profile at a low statistical uncertainty, and use the predicted refined dose profile to check against a dose profile of a radiotherapy treatment plan computed using one other dose algorithm.

At step 810, a radiotherapy treatment plan can be received with patient anatomical data and computed dose using one other dose algorithm, such as using a treatment planning system (TPS) implemented in the processor 114 of the radiotherapy system 100, which may include a radiation device to provide radiotherapy to a subject according to a treatment plan. At 820, a preliminary dose profile can be determined using the radiotherapy treatment plan and patient anatomical data, as similarly discussed above with respect to step 730 of the method 700. At 830, a refined dose profile can be predicted from the preliminary dose profile produced, as similarly discussed above with respect to step 740 of the method 700. At step 840, the refined dose profile can be compared to the dose of the radiotherapy treatment plan computed using one other dose algorithm. The comparison result can be provided to a user, who can then decide whether the refined dose profile can be delivered to the radiotherapy treatment plan which can be further be used to generate a radiotherapy for delivery to the patient.

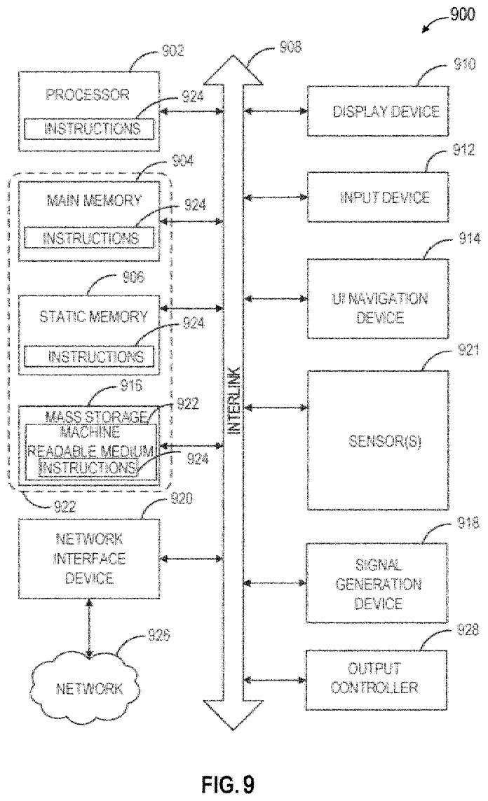

FIG. 9 illustrates a block diagram of an embodiment of a machine 900 on which one or more of the methods as discussed herein can be implemented. In one or more embodiments, one or more items of the data processing device 112 can be implemented by the machine 900. In alternative embodiments, the machine 900 operates as a standalone device or may be connected (e.g., networked) to other machines. In one or more embodiments, the data processing device 112 may include one or more of the items of the machine 900. In a networked deployment, the machine 900 may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example machine 900 includes a processor 902 (e.g., a central processing unit (CPU), a graphics processing unit (GPU), an application specific integrated circuit, circuitry, such as one or more transistors, resistors, capacitors, inductors, diodes, logic gates, multiplexers, buffers, modulators, demodulators, radios (e.g., transmit or receive radios or transceivers), sensors 921 (e.g., a transducer that converts one form of energy (e.g., light, heat, electrical, mechanical, or other energy) to another form of energy), or the like, or a combination thereof), a main memory 904 and a static memory 906, which communicate with each other via a bus 908. The machine 900 (e.g., computer system) may further include a video display unit 910 (e.g., a liquid crystal display (LCD) or a cathode ray tube (CRT)). The machine 900 also includes an alphanumeric input device 912 (e.g., a keyboard), a user interface (UI) navigation device 914 (e.g., a mouse), a disk drive or mass storage unit 916, a signal generation device 918 (e.g., a speaker) and a network interface device 920.

The disk drive unit 916 includes a machine-readable medium 922 on which is stored one or more sets of instructions and data structures (e.g., software) 924 embodying or utilized by any one or more of the methodologies or functions described herein. The instructions 924 may also reside, completely or at least partially, within the main memory 904 and/or within the processor 902 during execution thereof by the machine 900, the main memory 904 and the processor 902 also constituting machine-readable media.

The machine 900 as illustrated includes an output controller 928. The output controller 928 manages data flow to/from the machine 900. The output controller 928 is sometimes called a device controller, with software that directly interacts with the output controller 928 being called a device driver.

While the machine-readable medium 922 is shown in an embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), Electrically Erasable Programmable Read-Only Memory (EEPROM), and flash memory devices; magnetic disks such as internal hard disks and removable disks; magneto-optical disks; and CD-ROM and DVD-ROM disks.

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a local area network (“LAN”), a wide area network (“WAN”), the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended aspects, the terms “including” and “in which” are used as the plain-English equivalents of the respective terms “comprising” and “wherein.” Also, in the following aspects, the terms “comprising,” “including,” and “having” are intended to be open-ended to mean that there may be additional elements other than the listed elements, such that after such a term (e.g., comprising, including, having) in a aspect are still deemed to fall within the scope of that aspect. Moreover, in the following aspects, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples may include a computer-readable medium or machine-readable medium encoded with instructions operable to configure an electronic device to perform methods as described in the above examples. An implementation of such methods may include software code, such as microcode, assembly language code, a higher-level language code, or the like (e.g., “source code”). Such software code may include computer readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., read only memories (ROMs)), EPROMS, EEPROMS, or any type of media suitable for storing electronic instructions, and the like. Such computer readable storage medium coupled to a computer system bus to be accessible by the processor and other parts of the OIS.

In an embodiment, the computer-readable storage medium may have encoded a data structure for a treatment planning, wherein the treatment plan may be adaptive. The data structure for the computer-readable storage medium may be at least one of a Digital Imaging and Communications in Medicine (DICOM) format, an extended DICOM format, a XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, C, C++, Java, Python, and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended aspects. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended aspects, along with the full scope of equivalents to which such aspects are entitled.

Also, in the above Detailed Description, various features may be grouped together to streamline the disclosure. This should not be interpreted as intending that an unexpected disclosed feature is essential to any aspect. Rather, inventive subject matter may lie in less than all features of a particular disclosed embodiment. Thus, the following aspects are hereby incorporated into the Detailed Description, with each aspect standing on its own as a separate embodiment. The scope of the disclosure should be determined with reference to the appended aspects, along with the full scope of equivalents to which such aspects are entitled. Further, the limitations of the following aspects are not written in means-plus-function format and are not intended to be interpreted based on 35 U.S.C. § 112, sixth paragraph, unless and until such aspect limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the aspects.

1. A system for providing radiotherapy to a patient according to a treatment plan, the system comprising:
a memory configured to store a computational model;
a dose prediction engine configured to:
execute a dose simulation to calculate a preliminary dose profile at a first statistical uncertainty level of dose calculation; and
apply the calculated preliminary dose profile to the computational model to predict a refined dose profile at a second statistical uncertainty level, the second statistical uncertainty level being lower than the first statistical uncertainty level; and
a treatment planning system configured to generate or update a radiotherapy treatment plan based at least in part on the predicted refined dose profile.

2. The system of claim 1, wherein the preliminary dose profile and the predicted refined dose profile each include respective dose images representing spatial dose distributions at a radiotherapy treatment area and a treatment exclusion area.

3. The system of any of claim 1, wherein the preliminary dose profile and the predicted refined dose profile are each represented by at least one of:
a percentage depth dose (PDD) curve;
a radial dose curve;
a dose-volume histogram;
an overlap volume histogram; or
a three-dimensional dose distribution.

4. The system of claim 1, wherein the dose prediction engine is configured to predict the refined dose profile further using patient information including at least one of a particle density or a planning target volume (PTV) structure.

5. The system of claim 1, wherein the dose prediction engine is configured to calculate the preliminary dose profile using (i) patient anatomical data corresponding to a mapping of at least a radiotherapy treatment area and (ii) a dose calculation algorithm.

6. The system of claim 5, wherein the dose calculation algorithm includes a Monte Carlo (MC) algorithm or a collapsed cone convolution (CCC) algorithm.

7. The system of claim 5, wherein:
the dose prediction engine is configured to calculate the preliminary dose profile using an initial dose radiotherapy treatment plan; and
the treatment planning system is configured to update the initial radiotherapy treatment plan when the predicted refined dose profile satisfies a dose criterion.

8. The system of claim 1, wherein the computational model includes a trained deep-learning (DL) model, the system further comprising a training module configured to:
construct training data obtained from prior dose simulations on a radiotherapy treatment area, the training data comprising (i) a first set of dose profiles at the first statistical uncertainty level, and (ii) a second set of dose profiles at the second statistical uncertainty level lower than the first statistical uncertainty level; and
generate the trained DL model using the constructed training data, the trained DL model representing an established mapping from the first set of dose profiles to the second set of dose profiles for the same radiotherapy treatment area,
wherein the dose prediction engine is configured to apply the preliminary dose profile to the trained DL model to predict the refined dose profile.

9. The system of claim 8, wherein the trained DL model includes a convolutional neural network (CNN) with a U-Net architecture, the U-Net architecture including at least one of:
a standard U-Net;
a Hierarchy Dense (HD) U-Net;
an Attention U-Net;
a Group Normalization U-Net; or
a Recurrent Residual U-Net.

10. The system of claim 8, wherein the training module is configured to evaluate a model performance during model training, and to generate the trained DL model in response to the model performance satisfying a specific criterion, the model performance including at least one of:
a dose difference metric;
a radiotherapy planning target volume (PTV) dose coverage metric;
a gamma passing ratio; or
a structure similarity.

11. The system of claim 8, wherein the training data further comprises a third set of dose profiles obtained from the prior dose simulations on the radiotherapy treatment area, the a third set of dose profiles at a third statistical uncertainty level different from the first statistical uncertainty level and higher than the second statistical uncertainty level,
wherein the training module is configured to aggregate the first set of dose profiles with the third set of dose profiles, and to generate the trained DIL, model using the constructed training data including the aggregated first and third sets of dose profiles, the trained DL model representing an established mapping from the aggregated first and third sets of dose profiles to the second set of dose profiles.

12. The system of claim 11, wherein to predict the refined dose profile, the dose prediction engine is configured to:
execute a dose stimulation to determine a first preliminary dose profile at the first statistical uncertainty level and a second preliminary dose profile at the third statistical uncertainty level;
aggregate the first preliminary dose profile with the second preliminary dose profile; and
apply the aggregated first and second preliminary dose profiles to the trained DL model to predict the refined dose profile at the second statistical uncertainty level.

13. The system of claim 8, wherein the training data further comprises an intermediate set of dose profiles obtained from the prior dose simulations on the radiotherapy treatment area at an intermediate statistical uncertainty level lower than the first statistical uncertainty level and higher than the second statistical uncertainty level,
wherein the training module is configured to generate the trained DL model using the constructed training data including the intermediate set of dose profiles, the trained DL model including (i) a first trained DL model being trained to establish a mapping from the first set of dose profiles to the intermediate set of dose profiles and (ii) a second trained DL model being trained to establish a mapping from the intermediate set of dose profiles to the second set of dose profiles.

14. The system of claim 13, wherein to predict the refined dose profile, the dose prediction engine is configured to:
apply the preliminary dose profile to the first trained DL model to predict an intermediate dose profile at the intermediate statistical uncertainty level; and
apply the predicted intermediate dose profile to the second trained DL model to predict the refined dose profile at the second statistical uncertainty level.

15. The system of claim 1, wherein the dose prediction engine is configured to:
pre-process the preliminary dose profile including to down-sample, or to truncate at least a portion of, the preliminary dose profile; and
apply the pre-processed the preliminary dose profile to the computational model to predict the refined dose profile.

16. The system of claim 1, wherein the dose prediction engine is configured to post-process the predicted refined dose profile including to up-sample, or to interpolate or extrapolate at least a portion of, the predicted refined dose profile.

17. The system of claim 1, comprising a user interface configured to present the predicted refined dose profile or the generated or updated radiotherapy treatment plan to the user.

18. The system of claim 1, comprising a radiotherapy device configured to deliver a radiotherapy to the patient in accordance with the generated or updated radiotherapy treatment plan.

19. A method of providing radiotherapy according to a treatment plan, the method comprising:
calculating a preliminary dose profile at a first statistical uncertainty level of dose calculation in a dose simulation;
predicting a refined dose profile at a second statistical uncertainty level by applying the calculated preliminary dose profile to a computational model, the second statistical uncertainty level being lower than the first statistical uncertainty level; and
generating or updating a radiotherapy treatment plan based at least in part on the predicted refined dose profile.

20. The method of claim 19, wherein the preliminary dose profile and the predicted refined dose profile each include respective dose images representing spatial dose distributions at a radiotherapy treatment area and a treatment exclusion.

21. The method of claim 19, wherein determining the preliminary dose profile includes using (i) patient anatomical data corresponding to a mapping of at least a radiotherapy treatment area and (ii) a dose calculation algorithm.

22. The method of claim 21, wherein determining the preliminary dose profile further includes using an initial dose radiotherapy treatment plan,
wherein generating or updating the radiotherapy treatment plan includes updating the initial radiotherapy treatment plan when the predicted refined dose profile satisfies a dose criterion.

23. The method of claim 19, wherein the computational model includes a trained deep-learning (DL) model, the method further comprising:
constructing training data obtained from prior dose simulations on a radiotherapy treatment area, the training data comprising (i) a first set of dose profiles at the first statistical uncertainty level, and (ii) a second set of dose profiles at the second statistical uncertainty level lower than the first statistical uncertainty level; and
training a DL model using the constructed training data, the trained DL model representing an established mapping from the first set of dose profiles to the second set of dose profiles for the same radiotherapy treatment area,
wherein predicting the refined dose profile includes applying the calculated preliminary dose profile to the trained DL model.

24. The method of claim 23, further comprising evaluating a model performance, and generating the trained DL model in response to the model performance satisfying a specific criterion, the model performance including at least one of:
a dose difference metric;
a radiotherapy planning target volume (PTV) dose coverage metric;
a gamma passing ratio; or
a structure similarity.

25. The method of claim 23, wherein the trained DL model includes a convolutional neural network (CNN) with a U-Net architecture including at least one of a standard U-Net, a Hierarchy Dense (HD) U-Net, an Attention U-Net, a Group Normalization U-Net, or a Recurrent Residual U-Net.

26. The method of claim 23, wherein the training data further comprises a third set of dose profiles obtained from the prior dose simulations on the radiotherapy treatment area, the third set of dose profiles at a third statistical uncertainty level different from the first statistical uncertainty level and higher than the second statistical uncertainty level, the method further comprising:
aggregating the first set of dose profiles with the third set of dose profiles; and
training the DL model using the constructed training data including the aggregated first and third sets of dose profiles, the trained DL model representing an established mapping from the aggregated first and third sets of dose profiles to the second set of dose profiles.

27. The method of claim 26, wherein predicting the refined dose profile at the second statistical uncertainty level includes:
executing a dose stimulation to determine a first preliminary dose profile at the first statistical uncertainty level and a second preliminary dose profile at the third statistical uncertainty level;
aggregating the first preliminary dose profile with the second preliminary dose profile; and
applying the aggregated first and second preliminary dose profiles to the trained DL model to predict the refined dose profile.

28. The method claim 23, wherein the training data further comprises an intermediate set of dose profiles obtained from the prior dose simulations on the radiotherapy treatment area at an intermediate statistical uncertainty level lower than the first statistical uncertainty level and higher than the second statistical uncertainty level,
wherein training the DL model includes training a first DL model to establish a mapping from the first set of dose profiles to the intermediate set of dose profiles, and training a second DL model to establish a mapping from the intermediate set of dose profiles to the second set of dose profiles.

29. The method of claim 28, wherein predicting the refined dose profile at the second statistical uncertainty level includes:
applying the preliminary dose profile to the first trained DL model to predict an intermediate dose profile at the intermediate statistical uncertainty level; and
applying the predicted intermediate dose profile to the second trained DL model to predict the refined dose profile at the second statistical uncertainty level.

30. The method of claim 19, comprising delivering a radiotherapy in accordance with the generated or updated radiotherapy treatment plan using a radiotherapy device.
